(19) United States
(12) Patent Application Publication
(10) Pub. No.: US 2024/0016811 A1
(43) Pub. Date: Jan. 18, 2024

(54) CANCER THERAPY USING A COMBINATION OF CDK7 INHIBITOR WITH AN ANTI-MICROTUBULE AGENT

(71) Applicant: Aurigene Oncology Limited, Karnataka (IN)

(72) Inventors: Akhil Kumar, Karnataka (IN); Kishore Narayanan, Karnataka (IN); Ramulu Poddutoori, Karnataka (IN)

(21) Appl. No.: 18/032,919

(22) PCT Filed: Oct. 22, 2021
(86) PCT No.: PCT/IB2021/059751

§ 371 Date: Apr. 20, 2023

(30) Foreign Application Priority Data
Oct. 22, 2020 (IN) 202041046083

(51) Int. Cl. A61K 31/5377; A61K 31/337; A61P 35/00

(52) U.S. Cl. CPC A61K 31/5377; A61P 35/00; A61K 31/337

(57) ABSTRACT

The present invention provides a combination of a substituted heterocyclyl derivative of formula (I) or a pharmaceutically acceptable salt thereof which is a CDK7 inhibitor along with an anti-microtubule agent or prodrug thereof, and a method of treating or preventing cancer in a subject, comprising administering to the subject a compound of formula (I) conjointly with the anti-microtubule agent.

This application claims the benefit of Indian provisional application number 202041046083, filed on Oct. 22, 2020; the specifications of which are hereby incorporated by reference in their entirety.

### FIELD OF THE INVENTION

This invention pertains to methods for treating or preventing cancer in a subject, comprising administering to the subject a compound of formula (I) conjointly with an anti-microtubule agent; and related to combination therapy of a compound of formula (I) or a pharmaceutically acceptable salt or a stereoisomer thereof, and an anti-microtubule agent, derivative or prodrug thereof.

### BACKGROUND

Cyclin-dependent kinase (CDK)7 is necessary for transcription and acts by phosphorylating the C-terminal domain (CTD) of RNA polymerase II (PolII) to enable transcription initiation. CDK7 also modulates regulated gene expression by phosphorylating transcription factors, including p53, retinoid receptors, androgen receptor, and estrogen receptor. Ligand-dependent phosphorylation of serine 118 (Ser118), important for ERα function and turnover, is mediated by CDK7 (Oncogene; 2002; 21:4921). Hence pharmacological modulation of CDK7 kinase activity is considered to be an important approach to treat cancers that are critically dependent on transcription to maintain their oncogenic state. CDK7, which complexes with cyclin H and MAT1, phosphorylates the cell cycle CDKs in the activation of T-loop, to promote their activities (Fisher et al., Cell., August 26; 78(4):713-24, 1994). Recent genetic and biochemical studies have confirmed the importance of CDK7 for cell cycle progression (Larochelle et al., Mol Cell., March 23; 25(6):839-50. 2007; Ganuza et al., EMBO J., May 30; 31(11): 2498-510, 2012).

Prostate cancer is among the top three diagnosed cancers and the second leading cause of cancer deaths in men across the world (CA: Cancer J Clin., 2018; 68:394-424). Androgens acting through Androgen receptor (AR) are required for normal prostate development and function.

AR has three main functional domains: The N-terminal transcriptional regulation domain, the DNA binding domain (DBD) and the ligand binding domain. The N-terminal domain is the most variable, whilst the DBD is the most highly conserved (Clin Biochem Rev., 2016; 37(1):3-15). The Androgen receptor (AR) is a ligand-dependent transcription factor that upon binding to the its ligand (DHT), undergoes a conformational change leading to homodimerization, nuclear translocation and upregulation of gene transcription (Angew Chem Int Ed Engl., 2015; 54(33):9659-9662). The androgens Testosterone and Dihydrotestosterone (DHT) bind to AR and induce AR transcriptional activity. They promote the recruitment of cofactors following which AR trans locates to the nucleus and binds to AREs in the promoter regions of target genes to induce cell proliferation (Endocrine Reviews, 2004; 25(2):276-308). AR-mediated gene expression remains an important driver throughout prostate cancer progression. Most of the compounds available as therapies bind to the LBD of Androgen receptor. During early stages of Prostate cancer, antiandrogens such as bicalutamide are used as a first-line treatment. Although initially effective, the disease progresses to an androgen independent advanced stage called CRPC (castration resistant prostate cancer) which is responsible for the vast majority of prostate cancer deaths (Angew Chem Int Ed Engl., 2015; 54(33):9659-9662).

Targeting human AR in prostate cancers is an effective approach in metastatic and advanced cancers. 75-85% of patients who respond to first line of therapy initially, eventually develops resistance after few months of treatment. Mutations in the LBD region of AR leads to resistance resulting in CRPC. Studies have also identified presence of AR-Vs (AR splice variants) which confers resistance to cancer cells (Endocrine-Related Cancer, 2011; 18: R183-R196). The majority of these AR-Vs have NTD/DBD domains similar to the full-length AR but lack the LBD because of alternatively spliced 3′ terminal exons. These splice variants are ligand-independent forms of the AR and are constitutively active (Endocrinology, 2017; 158(6):1533-1542).

Although most men with metastatic prostate cancer initially respond to the historical standard-of-care, androgen-deprivation therapy, resistance inevitably develops after months to years, which is then known as metastatic castration-resistant prostate cancer (mCRPC). Resistance may be mediated by reactivation of androgen-receptor signaling through persistent adrenal androgen production, up-regulation of intratumoral testosterone production, modification of the biologic characteristics of androgen receptors, and steroidogenic parallel pathways [Yamaoka et ah, DOI: 10.1158/1078-0432.CCR-10-0255, 2010; Sharp et ah, J. Clin Invest, 129(1), 192-208, 2019; Armstrong and Lao, European Urology Oncology, Vol-2, 6, 677-684, 2018; Cao et al, Sci. Rep. 6, 25776, 2016; Vlachostergios et ah, Curr Oncol Rep. 2017 May; 19(5): 32. 2017; Wadosky and Koochekpour, Oncotarget. 2017; 8:18550-18576, 2017]. Despite the availability of multiple hormonal and non-hormonal agents, survival after the diagnosis of mCRPC remains limited. In addition, targeted approaches based on biomarkers have only recently emerged in this setting [Mateo et al, N Engl J Med 2015; 373:1697-1708, 2015].

Accordingly, there is a need for new treatments for cancer.

### SUMMARY

The invention provides methods and compositions for treating cancer using a compound of formula (I) or a pharmaceutically acceptable salt thereof, including combination therapy with an anti-microtubule agent.

Provided herein is a method of treating or preventing cancer in a subject, comprising administering to the subject a compound of formula (I):

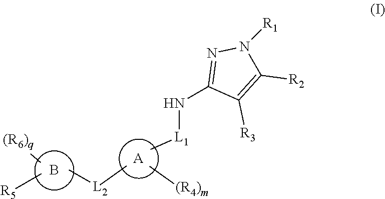

or a pharmaceutically acceptable salt or a stereoisomer thereof, conjointly with an anti-microtubule agent;
wherein,
ring A is cycloalkyl, aryl, heteroaryl or heterocyclyl;
ring B is aryl, cycloalkyl, heterocyclyl or absent;
R₁ is hydrogen or alkyl;
R₂ is hydrogen, alkyl or cycloalkyl;
R₃ is hydrogen, alkyl or heteroaryl;
alternatively, R₂ together with R₁ or R₃ along with the ring atoms to which they are attached forms a 5-7 membered ring;
R₄ at each occurrence is halo, alkyl, hydroxy, alkoxy, amino, nitro, cyano or haloalkyl;
R₅ is

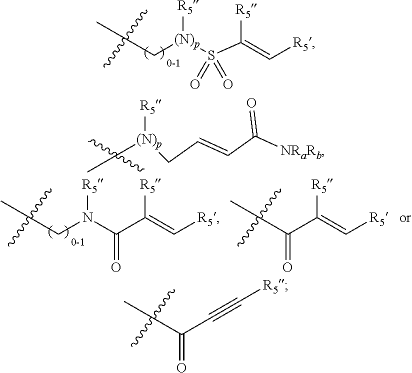

wherein R₅′ is hydrogen, halo, alkyl, alkoxy, alkoxyalkyl or —(CH₂)₁₋₃—NRₐR_{b}; R₅″ is H or alkyl;
Rₐ and R_{b} are each independently hydrogen, alkyl, alkoxy or alkoxyalkyl; alternatively, Rₐ and R_{b} together with the nitrogen atom to which they are attached form an optionally substituted ring containing 0-2 additional heteroatoms independently selected from N, O or S; wherein the optional substituent is one or more halo, alkyl, acyl, hydroxy, cyano, cyanoalkyl, haloalkyl, alkoxy, alkoxyalkyl, —COOH or —COO-alkyl;
R₆ at each occurrence is halo, alkyl, hydroxy, alkoxy, amino, nitro, cyano or haloalkyl;
L₁ is *—CR_{c}R_{d}—C(O)—, *—NRₑC(O)— or absent; wherein * is point of attachment with ring A;
R_{c} and R_{d} independently are hydrogen, alkyl or haloalkyl; alternatively, R_{c} and R_{d}together with the carbon to which they are attached form a cycloalkyl ring;
Rₑ is hydrogen or alkyl;
L₂ is —C(O)NH—, —C(O)O— or absent;
m is 0, 1 or 2;
p is 0 or 1; and
q is 0 to 3.

In some embodiments, disclosed herein is a use of a compound of formula (I) or a pharmaceutically acceptable salt or a stereoisomer thereof together with an anti-microtubule agent for the treatment and prevention of cancer.

In some embodiments, disclosed herein is a use of compound of formula (I) or a pharmaceutically acceptable salt or a stereoisomer thereof, including combinations thereof in all ratios, as a medicament for treating cancer conjointly with an anti-microtubule agent.

In some embodiments, the cancer is prostate cancer.

In some embodiments, the prostate cancer is metastatic castration-resistant prostate cancer.

In some embodiments, the prostate cancer is metastatic hormone resistant prostate cancer, castration naive prostate cancer, or castration resistant prostate cancer.

In some embodiments, the prostate cancer is metastatic hormone resistant prostate cancer.

In some embodiments, the prostate cancer is carcinoma of the prostate.

In some embodiments, the cancer is breast cancer, head and neck cancer, squamous cell carcinoma, stomach cancer, ovarian cancer, soft-tissue sarcoma, adenocarcinoma, lung cancer and non-small-cell lung cancer.

### BRIEF DESCRIPTION OF THE DRAWINGS

FIG. 1: Effect of docetaxel on proliferation of DU 145 cells

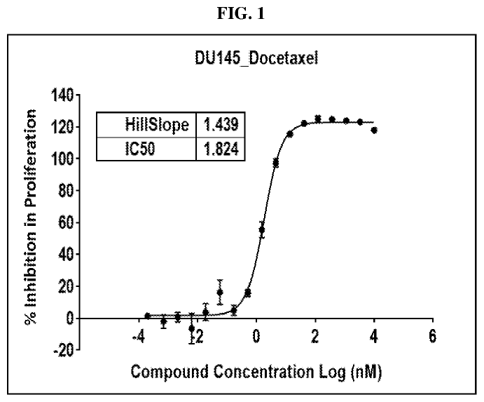

FIG. 2: Effect of Compound-44A on proliferation of DU 145 cells

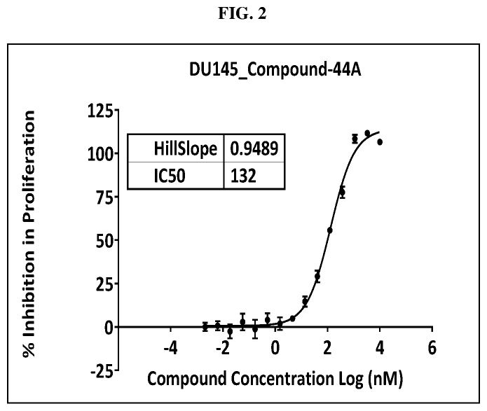

FIG. 3: Dose response shift of docetaxel in combination with Compound-44A in DU 145 cells

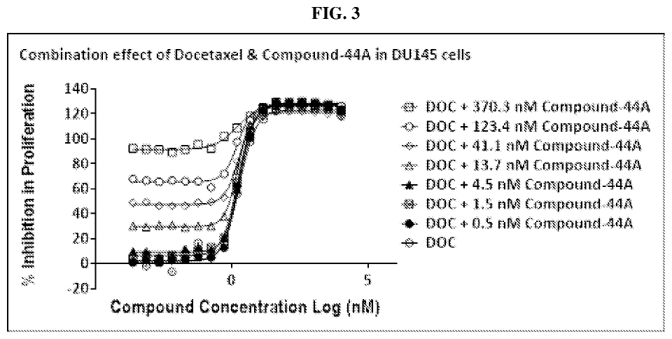

### DETAILED DESCRIPTION

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

As used herein, unless otherwise defined the term “alkyl” alone or in combination with other term(s) means saturated aliphatic hydrocarbon chains, including C₁-C₁₀ straight or C₃-C₁₀ branched alkyl groups. Examples of “alkyl” include but are not limited to methyl, ethyl, propyl, isopropyl, butyl, isobutyl, sec-butyl, tert-butyl, isopentyl or neopentyl and the like.

As used herein, the term “halo” or “halogen” alone or in combination with other term(s) means fluorine, chlorine, bromine or iodine.

As used herein, the term “haloalkyl” means alkyl substituted with one or more halogen atoms, wherein the alkyl groups are as defined above. The term “halo” is used herein interchangeably with the term “halogen” means F, Cl, Br or I. Examples of “haloalkyl” include but are not limited to fluoromethyl, difluoromethyl, chloromethyl, trifluoromethyl, 2,2,2-trifluoroethyl and the like.

As used herein, the term “hydroxy” or “hydroxyl” alone or in combination with other term(s) means —OH.

As used herein, the term “alkoxy” refers to the group alkyl-O— or —O-alkyl, where alkyl groups are as defined above. Exemplary alkoxy-groups include but are not limited to methoxy, ethoxy, n-propoxy, n-butoxy, t-butoxy and the like. An alkoxy group can be unsubstituted or substituted with one or more suitable groups.

As used herein, the term “alkoxyalkyl” refers to the group alkyl-O-alkyl-, wherein alkyl and alkoxy groups are as defined above. Exemplary alkoxyalkyl-groups include but are not limited to methoxymethyl, ethoxymethyl, methoxyethyl, isopropoxymethyl and the like.

As used herein, the term “cyano” refers to —CN; and the term “cyanoalkyl” refers to alkyl substituted with —CN; wherein the alkyl groups are as defined above.

As used herein, the term “amino” refers to —NH₂;

As used herein, the term “nitro” refers to —NO₂;

As used herein, the term “acyl” refers to the group —C(O)-alkyl, wherein alkyl groups are as defined above. Exemplary alkoxy-groups include but are not limited to acetyl, propanoyl and acrylyl. An alkoxy group can be unsubstituted or substituted with one or more suitable groups.

As used herein the term “cycloalkyl” alone or in combination with other term(s) means —C₃-C₁₀ saturated cyclic hydrocarbon ring. A cycloalkyl may be a single ring, which typically contains from 3 to 7 carbon ring atoms. Examples of single-ring cycloalkyls include but are not limited to cyclopropyl, cyclobutyl, cyclopentyl, cyclohexyl, cycloheptyl and the like. A cycloalkyl may alternatively be polycyclic or contain more than one ring. Examples of polycyclic cycloalkyls include bridged, fused and spirocyclyls and the like.

As used herein, the term “aryl” is optionally substituted monocyclic, bicyclic or polycyclic aromatic hydrocarbon ring system of about 6 to 14 carbon atoms. Examples of a C₆-C₁₄ aryl group include, but are not limited to phenyl, naphthyl, anthryl, tetrahydronaphthyl, fluorenyl, indanyl, biphenylenyl and acenaphthyl. Aryl group which can be unsubstituted or substituted with one or more suitable groups.

The term “heterocycloalkyl” refers to a non-aromatic, saturated or partially saturated monocyclic or polycyclic ring system of 3 to 15 members having at least one heteroatom or heterogroup selected from O, N, S, S(O), S(O)₂, NH or C(O) with the remaining ring atoms being independently selected from the group consisting of carbon, oxygen, nitrogen and sulfur. A monocyclic heterocycloalkyl may typically contain 4 to 7 ring atoms. Examples of “Heterocycloalkyl” include, but are not limited to azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, oxapiperazinyl, oxapiperidinyl, tetrahydrofuryl, tetrahydropyranyl, tetrahydrothiophenyl, dihydropyranyl, indolinyl, indolinylmethyl, azepanyl and N-oxides thereof. Attachment of a heterocycloalkyl substituent can occur via either a carbon atom or a heteroatom. A heterocycloalkyl group can be optionally substituted with one or more suitable groups by one or more aforesaid groups.

As used herein, the term “heteroaryl” alone or in combination with other term(s) means a completely unsaturated ring system containing a total of 5 to 14 ring atoms. At least one of the ring atoms is a heteroatom (i.e., oxygen, nitrogen, or sulfur), with the remaining ring atoms/groups being independently selected from the group consisting of carbon, oxygen, nitrogen or sulfur. A heteroaryl may be a single-ring (monocyclic) or polycyclic ring system. Examples of “heteroaryl” include but are not limited to pyridyl, indolyl, benzimidazolyl, benzothiazolyl and the like.

As used herein, the term “heterocyclyl” alone or in combination with other term(s) includes both “heterocycloalkyl” and “heteroaryl” groups which are as defined above.

The term “heteroatom” as used herein designates a sulfur, nitrogen or oxygen atom.

As used in the above definitions, the term “optionally substituted” or “substituted” or “optionally substituted with suitable groups” refers to replacement of one or more hydrogen radicals in a given structure with a radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl and heteroaryl. It is understood that the substituent may be further substituted.

As used herein, the term ‘compound(s)’ comprises the compounds disclosed in the present invention.

As used herein, the term “comprise” or “comprising” is generally used in the sense of include, that is to say permitting the presence of one or more features or components.

As used herein, the term “or” means “and/or” unless stated otherwise.

As used herein, the term “including” as well as other forms, such as “include”, “includes” and “included” is not limiting.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The term “treatment”/“treating” means any treatment of a disease in a mammal, including: (a) Inhibiting the disease, i.e., slowing or arresting the development of clinical symptoms; and/or (b) Relieving the disease, i.e., causing the regression of clinical symptoms and/or (c) Alleviating or abrogating a disease and/or its attendant symptoms.

As used herein, the term “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

“Pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

The term “pharmaceutically acceptable salt” refers to a product obtained by reaction of the compound of the present invention with a suitable acid or a base. Pharmaceutically acceptable salts of the compounds of this invention include those derived from suitable inorganic bases such as Li, Na, K, Ca, Mg, Fe, Cu, Al, Zn and Mn salts; Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts, and the like. Certain compounds of the invention (can form pharmaceutically acceptable salts with various organic bases such as lysine, arginine, guanidine, diethanolamine or metformin. Suitable base salts include, but are not limited to, aluminum, calcium, lithium, magnesium, potassium, sodium, or zinc, salts.

The term “stereoisomers” refers to any enantiomers, diastereoisomers, or geometrical isomers of the compounds of formula (I), (IA), (IB), (IC), (ID), (IE), (IF) and (IG), wherever they are chiral or when they bear one or more double bonds. When the compounds of the formula (I), (IA), (IB), (IC), (ID), (IE), (IF) and (IG) are chiral, they can exist in racemic or in optically active form. It should be understood that the invention encompasses all stereochemical isomeric forms, including diastereomeric, enantiomeric and epimeric forms, as well as d-isomers and l-isomers and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis/trans, syn/anti, entgegen (E)/zusammen (Z), (R)/(S) isomers as well as the appropriate mixtures thereof.

In certain embodiments, the compounds of the present invention can also contain unnatural proportions of atomic isotopes at one or more of the atoms that constitute such compounds. For example, the present invention also embraces isotopically-labeled variants of the present invention which are identical to those recited herein, but for the fact that one or more atoms of the compound are replaced by an atom having the atomic mass or mass number different from the predominant atomic mass or mass number usually found in nature for the atom. All isotopes of any particular atom or element as specified are contemplated within the scope of the compounds of the invention, and their uses. Exemplary isotopes that can be incorporated in to compounds of the invention include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds of the present inventions can generally be prepared by following procedures well known in the art, such as by substituting an isotopically labeled reagent for a non-isotopically labeled reagent.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. The examples of carriers, stabilizers and adjuvant are mentioned in literature like, Martin, Remington's Pharmaceutical Sciences, 15th Ed., Mack Publ. Co., Easton, PA [1975].

As used herein, the term “subject” refers to an animal, preferably a mammal, and most preferably a human.

As used herein, the term, “therapeutically effective amount” refers to an amount of a compound of the present invention or a pharmaceutically acceptable salt or a stereoisomer thereof; or a composition comprising the compound of the present invention or a pharmaceutically acceptable salt or a stereoisomer thereof, effective in producing the desired therapeutic response in a particular patient suffering from cancer. Particularly, the term “therapeutically effective amount” includes the amount of the compound of the present invention or a pharmaceutically acceptable salt or a stereoisomer thereof, when administered, that induces a positive modification in the disease or disorder to be treated or is sufficient to prevent development of, or alleviate to some extent, one or more of the symptoms of the disease or disorder being treated in a subject. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed the particular pharmaceutically acceptable carrier utilized.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. In some embodiments, the additional therapeutic compound is administered within about 5 minutes to within about 168 hours prior to or after administration of a compound of formula (I). Thus, a subject who receives such treatment can benefit from a combined effect of different therapeutic compounds.

In some embodiments, the present invention provides methods wherein docetaxel is administered within about 5 minutes to within about 168 hours prior to or after administration of a compound of formula (I). In some embodiments, the present invention provides methods wherein docetaxel is administered within 0.5 hours to 72 hours prior to or after administration of a compound of formula (I). In some embodiments, the present invention provides methods wherein docetaxel is administered within 0.5 hours to 24 hours prior to or after administration of a compound of formula (I). In some embodiments, the present invention provides methods wherein docetaxel is administered within 2 hours prior to or after administration of a compound of formula (I).

In some embodiments, the present invention provides methods comprising administering a compound of formula (I) prior to administering docetaxel. In some embodiments, the present invention provides methods comprising administering a compound of formula (I) 2 hours prior to administering docetaxel. In some embodiments, the present invention provides methods comprising administering a compound of formula (I) within 0.5 hours to 72 hours after administering docetaxel. In some embodiments, the present invention provides methods comprising administering a compound of formula (I) about 2 hours after administering docetaxel. In some embodiments, the present invention provides methods comprising administering a compound of formula (I) at least 1 hour after docetaxel administration.

In certain embodiments, conjoint administration of compounds of the invention with one or more additional therapeutic agent(s) (e.g., one or more additional chemotherapeutic agent(s)) provides improved efficacy relative to each individual administration of the compound of the invention or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

Provided herein is a method of treating or preventing cancer in a subject, comprising administering to the subject a compound of Formula (I):

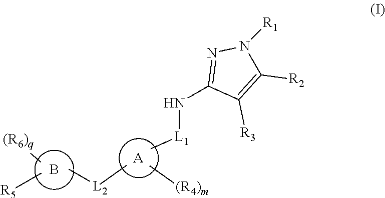

or a pharmaceutically acceptable salt or a stereoisomer thereof, conjointly with an anti-microtubule agent;
wherein,
ring A is cycloalkyl, aryl, heteroaryl or heterocyclyl;
ring B is aryl, cycloalkyl, heterocyclyl or absent;
R₁ is hydrogen or alkyl;
R₂ is hydrogen, alkyl or cycloalkyl;
R₃ is hydrogen, alkyl or heteroaryl;
alternatively, R₂ together with R₁ or R₃ along with the ring atoms to which they are attached forms a 5-7 membered ring;
R₄ at each occurrence is halo, alkyl, hydroxy, alkoxy, amino, nitro, cyano or haloalkyl;
R₅ is

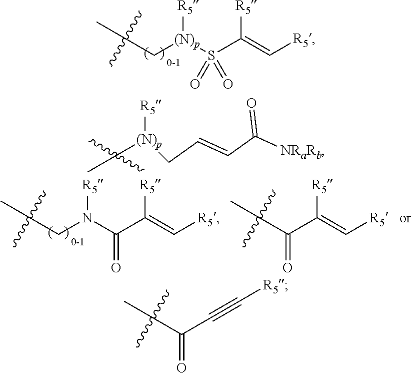

wherein R₅′ is hydrogen, halo, alkyl, alkoxy, alkoxyalkyl or —(CH₂)₁₋₃—NRₐR_{b}; R₅″ is H or alkyl;
Rₐ and R_{b} are each independently hydrogen, alkyl, alkoxy or alkoxyalkyl; alternatively, Rₐ and R_{b} together with the nitrogen atom to which they are attached form an optionally substituted ring containing 0-2 additional heteroatoms independently selected from N, O or S; wherein the optional substituent is one or more halo, alkyl, acyl, hydroxy, cyano, cyanoalkyl, haloalkyl, alkoxy, alkoxyalkyl, —COOH or —COO-alkyl;
R₆ at each occurrence is halo, alkyl, hydroxy, alkoxy, amino, nitro, cyano or haloalkyl;
L₁ is *—CR_{c}R_{d}—C(O)—, *—NRₑC(O)— or absent; wherein * is point of attachment with ring A;
R_{c} and R_{d} independently are hydrogen, alkyl or haloalkyl; alternatively, R_{c} and R_{d} together with the carbon to which they are attached form a cycloalkyl ring;
Rₑ is hydrogen or alkyl;
L₂ is —C(O)NH—, —C(O)O— or absent;
m is 0, 1 or 2;
p is 0 or 1; and
q is 0 to 3.

In some embodiments, the present methods include a compound of formula (I) represented by formula (IA):

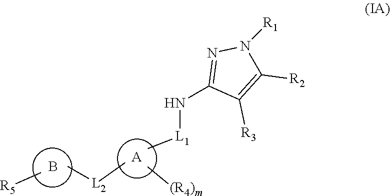

or a pharmaceutically acceptable salt thereof or a stereoisomer thereof;
wherein,
ring A is cycloalkyl, aryl, heteroaryl or heterocyclyl;
ring B is aryl, cycloalkyl, heterocyclyl or absent;
R₁ is hydrogen or alkyl;
R₂ is hydrogen, alkyl or cycloalkyl;
R₃ is hydrogen, alkyl or heteroaryl;
alternatively, R₂ together with R₁ or R₃ along with the ring atoms to which they are attached forms a 5-7 membered ring;
R₄ at each occurrence is halo, alkyl, hydroxy or alkoxy;
R₅ is

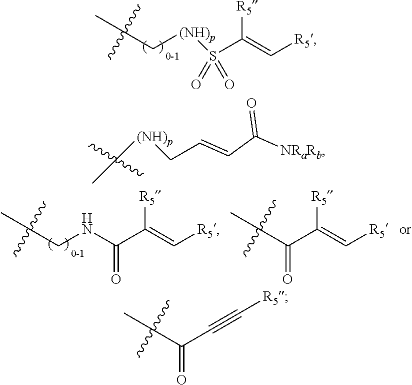

wherein R₅′ is hydrogen, halo, alkyl, alkoxy, alkoxyalkyl or —(CH₂)₁₋₃—NRₐR_{b}; R₅″ is H or alkyl;
Rₐ and R_{b} are each independently hydrogen or alkyl; alternatively, Rₐ and R_{b} together with the nitrogen atom to which they are attached form an optionally substituted ring containing 0-2 additional heteroatoms independently selected from N, O or S; wherein the optional substituent is one or more halo, alkyl, hydroxy, haloalkyl or alkoxy;
L₁ is *—CR_{c}R_{d}—C(O)—, *—NRₑC(O)— or absent; wherein * is point of attachment with ring A;
R_{c} and R_{d} independently are hydrogen, alkyl or haloalkyl; alternatively, R_{c} and R_{d} together with the carbon to which they are attached form a cycloalkyl ring;
Rₑ is hydrogen or alkyl;
L₂ is —C(O)NH—, —C(O)O— or absent;
m is 0, 1 or 2; and
p is 0 or 1.

In some embodiments, the present methods include a compound of formula (I) represented by formula (IB):

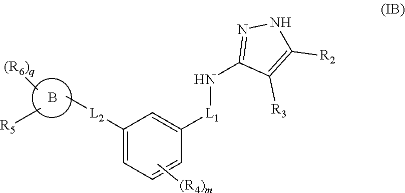

or a pharmaceutically acceptable salt thereof or a stereoisomer thereof.

In some embodiments, the present methods include a compound of formula (I) represented by formula (IC):

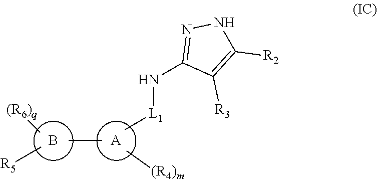

or a pharmaceutically acceptable salt thereof or a stereoisomer thereof.

In some embodiments, the present methods include a compound of formula (I) represented by formula (ID):

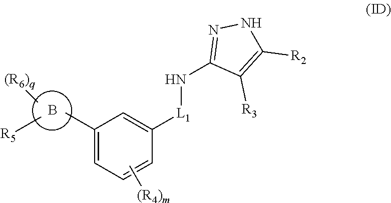

or a pharmaceutically acceptable salt thereof or a stereoisomer thereof.

In some embodiments, the present methods include a compound of formula (I) represented by formula (IE):

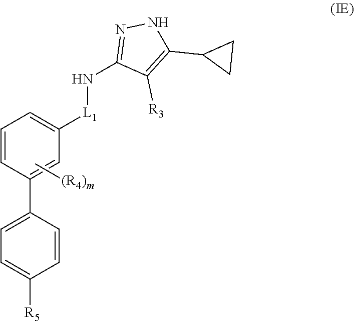

or a pharmaceutically acceptable salt thereof or a stereoisomer thereof;
wherein, L₁ is *—CR_{c}R_{d}—C(O)— or *—NRₑC(O)—; wherein * is point of attachment with phenyl ring.

In some embodiments, the present methods include a compound of formula (I) represented by formula (IF):

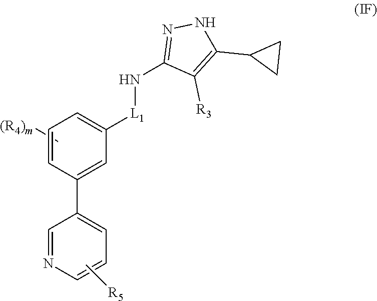

or a pharmaceutically acceptable salt thereof or a stereoisomer thereof;
wherein, L₁ is *—CR_{c}R_{d}—C(O)— or *—NRₑC(O)—; wherein * is point of attachment with phenyl ring.

In some embodiments, the present methods include a compound of formula (I) represented by formula (IG):

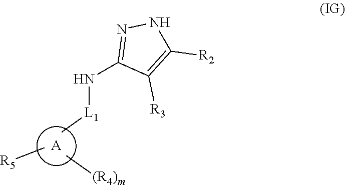

or a pharmaceutically acceptable salt thereof or a stereoisomer thereof.

In some embodiments, provided herein is a method of treating or preventing cancer in a subject, comprising administering to the subject a compound of formula (I):

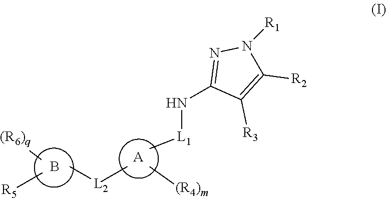

or a pharmaceutically acceptable salt or a stereoisomer thereof, conjointly with docetaxel;
wherein,
ring A is cycloalkyl, aryl, heteroaryl or heterocyclyl;
ring B is aryl, cycloalkyl, heterocyclyl or absent;
R₁ is hydrogen or alkyl;
R₂ is hydrogen, alkyl or cycloalkyl;
R₃ is hydrogen, alkyl or heteroaryl;
alternatively, R₂ together with R₁ or R₃ along with the ring atoms to which they are attached forms a 5-7 membered ring;
R₄ at each occurrence is halo, alkyl, hydroxy, alkoxy, amino, nitro, cyano or haloalkyl;
R₅ is

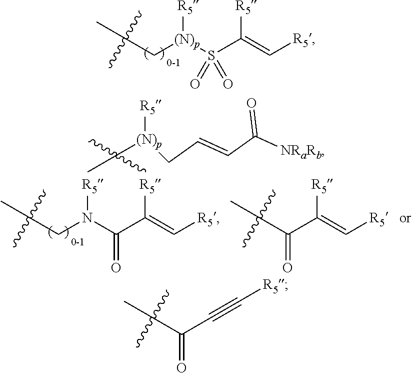

wherein R₅′ is hydrogen, halo, alkyl, alkoxy, alkoxyalkyl or —(CH₂)₁₋₃—NRₐR_{b}; R₅″ is H or alkyl;
Rₐ and R_{b} are each independently hydrogen, alkyl, alkoxy or alkoxyalkyl; alternatively, Rₐ and R_{b} together with the nitrogen atom to which they are attached form an optionally substituted ring containing 0-2 additional heteroatoms independently selected from N, O or S;

wherein the optional substituent is one or more halo, alkyl, acyl, hydroxy, cyano, cyanoalkyl, haloalkyl, alkoxy, alkoxyalkyl, —COOH or —COO-alkyl;
R₆ at each occurrence is halo, alkyl, hydroxy, alkoxy, amino, nitro, cyano or haloalkyl;
L₁ is *—CR_{c}R_{d}—C(O)—, *—NRₑC(O)— or absent; wherein * is point of attachment with ring A;
R_{c} and R_{d} independently are hydrogen, alkyl or haloalkyl; alternatively, R_{c} and R_{d} together with the carbon to which they are attached form a cycloalkyl ring;
Rₑ is hydrogen or alkyl;
L₂ is —C(O)NH—, —C(O)O— or absent;
m is 0, 1 or 2;
p is 0 or 1; and
q is 0 to 3.

In some embodiments, the present methods include a compound of formula (I) wherein ring A is aryl or heteroaryl.

In some embodiments, the present methods include a compound of formula (I) or a pharmaceutically acceptable salt thereof, wherein ring A is aryl.

In some embodiments, the present methods include a compound of formula (I) or a pharmaceutically acceptable salt thereof, wherein ring A is 1,3-phenylene.

In some embodiments, the present methods include a compound of formula (I) or a pharmaceutically acceptable salt thereof, wherein ring B is monocyclic or bicyclic, cycloalkyl, aryl, heterocycloalkyl or heteroaryl.

According to another embodiment, specifically provided are compounds of formula (I), wherein ring B is aryl; preferably the said aryl is phenyl.

According to yet another embodiment, specifically provided are compounds of formula (I), wherein ring B is heterocyclyl; preferably the said heterocyclyl is piperidinyl, pyridinyl, piperazinyl, pyrazolyl, morpholinyl, indolinyl or pyrrolidinyl.

In some embodiments, the present methods include a compound of formula (I) or a pharmaceutically acceptable salt thereof, wherein R₂ is cycloalkyl.

In some embodiments, the present methods include a compound of formula (I) or a pharmaceutically acceptable salt thereof, wherein R₅ is

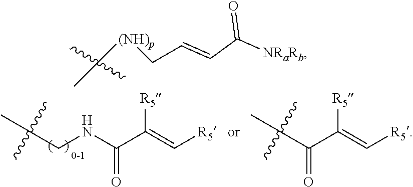

In some embodiments, the present methods include a compound of formula (I) or a pharmaceutically acceptable salt thereof, wherein R₅′ is —(CH₂)₁₋₃—NRₐR_{b}.

In some embodiments, the present methods include a compound of formula (I) or a pharmaceutically acceptable salt thereof, wherein wherein Rₐ and R_{b} together with the nitrogen atom to which they are attached form an optionally substituted heterocyclic ring having 0-2 additional heteroatoms selected from O, S or N.

In some embodiments, the present methods include a compound of formula (I) or a pharmaceutically acceptable salt thereof, wherein L₁ is *—CR_{c}R_{d}—C(O)—; wherein * is the point of attachment with ring A.

According to the preceding embodiment, specifically provided are methods which include a compound of formula (I), wherein R_{c} and R_{d}are independently hydrogen or alkyl, wherein the said alkyl is methyl, ethyl or isopropyl.

In some embodiments, the present methods include a compound of formula (I) or a pharmaceutically acceptable salt thereof, wherein L₂ is absent.

In some embodiments, the present methods include a compound of formula (I) or a pharmaceutically acceptable salt thereof, wherein when L₁ is present, L₂ is absent.

In some embodiments, the present methods include a compound of formula (I) or a pharmaceutically acceptable salt thereof, wherein R₂ is alkyl or cycloalkyl; preferably the said alkyl is ethyl and the said cycloalkyl is cyclopropyl, cyclobutyl or cyclopentyl.

In some embodiments, the present methods include a compound of formula (I) or a pharmaceutically acceptable salt thereof, wherein R₃ is hydrogen and alkyl; wherein the said alkyl is methyl.

In some embodiments, the present methods include a compound of formula (I) or a pharmaceutically acceptable salt thereof, wherein R₄ is halo; preferably the said halo is fluoro.

In some embodiments, the present methods include a compound of formula (I) or a pharmaceutically acceptable salt thereof, wherein R₅ is

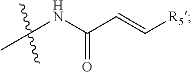

wherein R₅′ is hydrogen or —CH₂—NRₐR_{b}.

In some embodiments, the present methods include a compound of formula (I) or a pharmaceutically acceptable salt thereof, wherein R₅ is

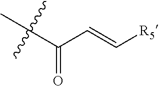

when R₅ is attached to hetero atom of ring B; R₅′ is hydrogen or —CH₂—NRₐR_{b}.

According to the preceding two embodiments, the present methods include a compound of formula (I) or a pharmaceutically acceptable salt thereof, wherein the said Rₐ and R_{b} are each independently hydrogen or alkyl; preferably the said alkyl is methyl.

According to the preceding embodiment, the present methods include a compound of formula (I) or a pharmaceutically acceptable salt thereof, wherein the said Rₐ and R_{b} together with the nitrogen to which they are attached form an optionally substituted ring containing 0-2 additional heteroatoms independently selected from N, O or S; wherein the optional substituent is one or more halo, hydroxy, haloalkyl or alkoxy.

In some embodiments, the present methods include a compound of formula (I) or a pharmaceutically acceptable salt thereof, wherein ring A is meta-substituted with respect to L₁ and L₂.

In some embodiments, the present methods include a compound of formula (I) or a pharmaceutically acceptable salt thereof, wherein ring B is monocyclic or bicyclic, cycloalkyl, aryl, heterocycloalkyl or heteroaryl.

In some embodiments, the present methods include a compound of formula (I) or a pharmaceutically acceptable salt thereof, wherein ring B is heterocyclyl; in another embodiment the said heterocyclyl is piperidinyl, pyridinyl,1,2,3,6-tetrahydropyridinyl, piperazinyl, pyrazinyl, pyrazolyl, morpholinyl, indolinyl or pyrrolidinyl.

In some embodiments, the present methods include a compound of formula (I) or a pharmaceutically acceptable salt thereof, wherein m is 0 or 1.

In some embodiments, the present methods include a compound of formula (I) or a pharmaceutically acceptable salt thereof, wherein m is 1.

In some embodiments, the present methods include a compound of formula (I) or a pharmaceutically acceptable salt thereof, wherein n is 1 or 2.

In some embodiments, the present methods include a compound of formula (I) or a pharmaceutically acceptable salt thereof, wherein n is 1.

In some embodiments, the present methods include a compound of formula (I) or a pharmaceutically acceptable salt thereof, wherein R₂ is cycloalkyl; in another embodiment, the said cycloalkyl is cyclopropyl, cyclobutyl or cyclopentyl.

In some embodiments, the present methods include a compound of formula (I) or a pharmaceutically acceptable salt thereof, wherein R₂ and R₁ together with the atoms to which they are attached form a 5 or 6 membered ring.

In some embodiments, the present methods include a compound of formula (I) or a pharmaceutically acceptable salt thereof, wherein R₂ and R₃ together with the atoms to which they are attached form a 5 or 6 membered ring.

In some embodiments, the present methods include a compound of formula (I) or a pharmaceutically acceptable salt thereof, wherein R₂ and R₃ together with the atoms to which they are attached form a 6 membered aromatic ring.

In some embodiments, the present methods include a compound of formula (I) or a pharmaceutically acceptable salt thereof, wherein R₅ is

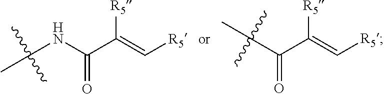

wherein R₅′ is hydrogen, halo, alkyl, alkoxy or alkoxyalkyl; and R₅″ is H or alkyl.

In some embodiments, the present methods include a compound of formula (I) or a pharmaceutically acceptable salt thereof, wherein R₅ is

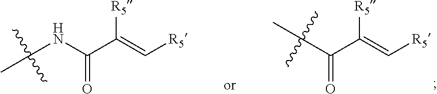

wherein R₅′ is —CH₂—NRₐR_{b}; R₅″ is H or alkyl; Rₐ and R_{b} are each independently hydrogen, alkyl, alkoxy or alkoxyalkyl.

In some embodiments, the present methods include a compound of formula (I) or a pharmaceutically acceptable salt thereof, wherein R₅ is

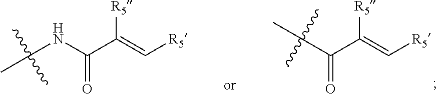

wherein R₅′ is — CH₂—NRₐR_{b}; R₅″ is H or alkyl; Rₐ and R_{b} together with the nitrogen atom to which they are attached form an optionally substituted 4-7 membered heterocyclyl ring containing 0-2 additional heteroatoms independently selected from N, O or S; wherein the optional substituent is one or more halo, alkyl, acyl, hydroxy, cyano, cyanoalkyl, haloalkyl, alkoxy, alkoxyalkyl, —COOH or —COO-alkyl.

In some embodiments, the present methods include a compound of formula (I) or a pharmaceutically acceptable salt thereof, wherein R₅ is

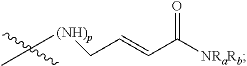

wherein p, Rₐ and R_{b} are as defined in formula (I).

In some embodiments, the present methods include a compound of formula (I) or a pharmaceutically acceptable salt thereof, wherein R₅ is,

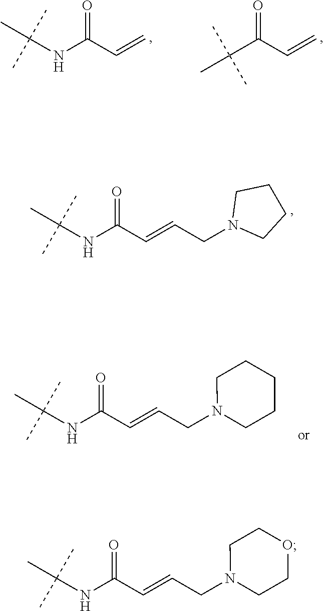

wherein [symbol] is a point of attachment.

In some embodiments, the present methods include a compound of formula (I) or a pharmaceutically acceptable salt thereof, wherein R₅′ is —(CH₂)₁₋₃—NRₐR_{b}; wherein Rₐ and R_{b} are as defined in formula (I) .

In some embodiments, the present methods include a compound of formula (I) or a pharmaceutically acceptable salt thereof, wherein when L₁ is —CR_{c}R_{d}—C(O)— or —NRₑC(O)—, L₂ is absent.

In some embodiments, the present methods include administering a compound of formula (I) selected from Table-1 below:

TABLE 1

| Compound | |
|---|---|
| No. | IUPAC name |
| 1 | (E)-N-(3′-(1-((5-cyclopropyl-1H-pyrazol-3-yl)amino)-1-oxopropan-2-yl)-[1,1′- |
| | biphenyl]-4-yl)-4-(dimethylamino)but-2-enamide; |
| 2 | (E)-N-(3′-(1-((5-cyclopropyl-1H-pyrazol-3-yl)amino)-1-oxopropan-2-yl)-[1,1′- |
| | biphenyl]-4-yl)-4-(dimethylamino)but-2-enamide (Isomer-1 of compound-1); |
| 3 | (E)-N-(3′-(1-((5-cyclopropyl-1H-pyrazol-3-yl)amino)-1-oxopropan-2-yl)-[1,1′- |
| | biphenyl]-4-yl)-4-(dimethylamino)but-2-enamide (Isomer-2 of compound-1); |
| 4 | (E)-N-(5′-(1-((5-cyclopropyl-1H-pyrazol-3-yl)amino)-1-oxopropan-2-yl)-2′-fluoro- |
| | [1,1′-biphenyl]-4-yl)-4-(dimethylamino)but-2-enamide; |
| 5 | (E)-N-(3′-(1-((5-cyclopropyl-1H-pyrazol-3-yl)amino)-1-oxopropan-2-yl)-4′-fluoro- |
| | [1,1′-biphenyl]-4-yl)-4-(dimethylamino)but-2-enamide; |
| 7 | (E)-N-(3′-(2-((5-cyclopropyl-1H-pyrazol-3-yl)amino)-2-oxoethyl)-[1,1′-biphenyl]-4- |
| | yl)-4-(dimethylamino)but-2-enamide; |
| 8 | (E)-4-(dimethylamino)-N-(3′-(1-((5-ethyl-1H-pyrazol-3-yl)amino)-1-oxopropan-2- |
| | yl)-[1,1′-biphenyl]-4-yl)but-2-enamide; |
| 9 | (E)-N-(3′-(1-((5-(tert-butyl)-1H-pyrazol-3-yl)amino)-1-oxopropan-2-yl)-[1,1′- |
| | biphenyl]-4-yl)-4-(dimethylamino)but-2-enamide; |
| 10 | (E)-N-(5-(3-(1-((5-cyclopropyl-1H-pyrazol-3-yl)amino)-1-oxopropan-2- |
| | yl)phenyl)pyridin-2-yl)-4-morpholinobut-2-enamide; |
| 11 | (E)-N-(5-(3-(1-((5-cyclopropyl-1H-pyrazol-3-yl)amino)-1-oxopropan-2- |
| | yl)phenyl)pyridin-2-yl)-4-morpholinobut-2-enamide (Isomer-1 of compound-10); |
| 12 | (E)-N-(5-(3-(1-((5-cyclopropyl-1H-pyrazol-3-yl)amino)-1-oxopropan-2- |
| | yl)phenyl)pyridin-2-yl)-4-morpholinobut-2-enamide (Isomer-2 of compound-10); |
| 13 | (E)-N-(3′-(1-((5-cyclopropyl-1H-pyrazol-3-yl)amino)-1-oxobutan-2-yl)-[1,1′- |
| | biphenyl]-4-yl)-4-(dimethylamino)but-2-enamide; |
| 14 | (E)-N-(3′-(1-((5-cyclopropyl-1H-pyrazol-3-yl)amino)-1-oxopropan-2-yl)-3-fluoro- |
| | [1,1′-biphenyl]-4-yl)-4-(dimethylamino)but-2-enamide; |
| 15 | (E)-N-(3′-(1-((5-cyclobutyl-1H-pyrazol-3-yl)amino)-1-oxopropan-2-yl)-[1,1′- |
| | biphenyl]-4-yl)-4-(dimethylamino)but-2-enamide; |
| 16 | (E)-N-(6-(3-(1-((5-cyclopropyl-1H-pyrazol-3-yl)amino)-1-oxopropan-2- |
| | yl)phenyl)pyridin-3-yl)-4-(dimethylamino)but-2-enamide; |
| 17 | (E)-N-(3′-(1-((5-cyclopropyl-1H-pyrazol-3-yl)amino)-1-oxopropan-2-yl)-2-fluoro- |
| | [1,1′-biphenyl]-4-yl)-4-(dimethylamino)but-2-enamide; |
| 18 | N-(3′-(1-((5-cyclopropyl-1H-pyrazol-3-yl)amino)-1-oxopropan-2-yl)-[1,1′- |
| | biphenyl]-4-yl)acrylamide; |
| 19 | N-(3′-(1-((5-cyclopropyl-1H-pyrazol-3-yl)amino)-1-oxopropan-2-yl)-3-fluoro-[1,1′- |
| | biphenyl]-4-yl)acrylamide; |
| 20 | N-(5-(3-(1-((5-cyclopropyl-1H-pyrazol-3-yl)amino)-1-oxopropan-2- |
| | yl)phenyl)pyridin-2-yl)acrylamide; |
| 21 | N-(5-(3-(1-((5-cyclopropyl-1H-pyrazol-3-yl)amino)-1-oxopropan-2- |
| | yl)phenyl)pyridin-2-yl)acrylamide (Isomer-1 of compound-20); |
| 22 | N-(5-(3-(1-((5-cyclopropyl-1H-pyrazol-3-yl)amino)-1-oxopropan-2- |
| | yl)phenyl)pyridin-2-yl)acrylamide (Isomer-2 of compound-20); |
| 23 | N-(5′-(1-((5-cyclopropyl-1H-pyrazol-3-yl)amino)-1-oxopropan-2-yl)-2′,3-difluoro- |
| | [1,1′-biphenyl]-4-yl)acrylamide; |
| 24 | N-(6-(3-(1-((5-cyclopropyl-1H-pyrazol-3-yl)amino)-1-oxopropan-2- |
| | yl)phenyl)pyridin-3-yl)acrylamide; |
| 25 | 2-(4′-acrylamido-3′-fluoro-[1,1′-biphenyl]-3-yl)-N-(5-cyclopropyl-1H-pyrazol-3-yl)- |
| | 3-methylbutanamide; |
| 26 | 2-(3-(5-acrylamidopyridin-2-yl)phenyl)-N-(5-cyclopropyl-1H-pyrazol-3- |
| | yl)butanamide; |
| 27 | 2-(4′-acrylamido-3′-fluoro-[1,1′-biphenyl]-3-yl)-N-(5-cyclopropyl-1H-pyrazol-3- |
| | yl)butanamide; |
| 28 | (E)-N-(3′-(1-((5-cyclopentyl-1H-pyrazol-3-yl)amino)-1-oxopropan-2-yl)-3-fluoro- |
| | [1,1′-biphenyl]-4-yl)-4-(dimethylamino)but-2-enamide; |
| 29 | (E)-N-(5-cyclopropyl-1H-pyrazol-3-yl)-2-(3-(1-(4-(dimethylamino)but-2- |
| | enoyl)indolin-5-yl)phenyl)propanamide; |
| 30 | N-(3′-(1-((5-cyclopropyl-4-methyl-1H-pyrazol-3-yl)amino)-1-oxopropan-2-yl)-[1,1′- |
| | biphenyl]-4-yl)acrylamide; |
| 31 | (E)-N-(3′-(1-((5-cyclopropyl-1H-pyrazol-3-yl)amino)-1-oxobutan-2-yl)-3-fluoro- |
| | [1,1′-biphenyl]-4-yl)-4-(dimethylamino)but-2-enamide; |
| 32 | (E)-N-(3′-(1-((5-cyclopropyl-1H-pyrazol-3-yl)amino)-1-oxopropan-2-yl)-3-fluoro- |
| | [1,1′-biphenyl]-4-yl)-4-(diethylamino)but-2-enamide; |
| 33 | (E)-N-(3′-(1-((5-cyclopropyl-1H-pyrazol-3-yl)amino)-1-oxopropan-2-yl)-3-fluoro- |
| | [1,1′-biphenyl]-4-yl)-4-(pyrrolidin-1-yl)but-2-enamide; |
| 34 | (E)-N-(3′-(1-((5-cyclopropyl-1H-pyrazol-3-yl)amino)-1-oxopropan-2-yl)-3-fluoro- |
| | [1,1′-biphenyl]-4-yl)-4-morpholinobut-2-enamide; |
| 35 | (E)-N-(6-(3-(1-((5-cyclopropyl-1H-pyrazol-3-yl)amino)-1-oxopropan-2- |
| | yl)phenyl)pyridin-3-yl)-4-morpholinobut-2-enamide; |
| 36 | (E)-N-(6-(3-(1-((5-cyclopropyl-1H-pyrazol-3-yl)amino)-3-methyl-1-oxobutan-2- |
| | yl)phenyl)pyridin-3-yl)-4-(dimethylamino)but-2-enamide; |
| 37 | (E)-4-((6-(3-(1-((5-cyclopropyl-1H-pyrazol-3-yl)amino)-1-oxopropan-2- |
| | yl)phenyl)pyridin-3-yl)amino)-N,N-dimethylbut-2-enamide; |
| 38 | (E)-N-(6-(3-(1-((5-cyclopropyl-1H-pyrazol-3-yl)amino)-1-oxopropan-2-yl)phenyl)- |
| | 2-fluoropyridin-3-yl)-4-(dimethylamino)but-2-enamide; |
| 39 | (E)-N-(6-(3-(1-((5-cyclopropyl-1H-pyrazol-3-yl)amino)-1-oxopropan-2- |
| | yl)phenyl)pyridin-3-yl)-4-(piperidin-1-yl)but-2-enamide; |
| 40 | (E)-N-(5-(3-(1-((5-cyclopropyl-1H-pyrazol-3-yl)amino)-1-oxopropan-2- |
| | yl)phenyl)pyridin-2-yl)-4-(piperidin-1-yl)but-2-enamide; |
| 41 | (E)-N-(5-(3-(1-((5-cyclopropyl-1H-pyrazol-3-yl)amino)-1-oxopropan-2- |
| | yl)phenyl)pyridin-2-yl)-4-methoxybut-2-enamide; |
| 42 | (E)-N-(5-(3-(1-((5-cyclopropyl-1H-pyrazol-3-yl)amino)-1-oxopropan-2- |
| | yl)phenyl)pyridin-2-yl)-4-(dimethylamino)but-2-enamide; |
| 43 | (E)-N-(3′-(1-((5-cyclopropyl-1H-pyrazol-3-yl)amino)-3-methyl-1-oxobutan-2-yl)-3- |
| | fluoro-[1,1′-biphenyl]-4-yl)-4-(dimethylamino)but-2-enamide; |
| 44 | (E)-N-(5-(3-(1-((5-cyclopropyl-1H-pyrazol-3-yl)amino)-3-methyl-1-oxobutan-2- |
| | yl)phenyl)pyridin-2-yl)-4-morpholinobut-2-enamide; |
| 44A | (S)-(E)-N-(5-(3-(1-((5-cyclopropyl-1H-pyrazol-3-yl)amino)-3-methyl-1-oxobutan-2- |
| | yl)phenyl)pyridin-2-yl)-4-morpholinobut-2-enamide; |
| 44B | (R)-(E)-N-(5-(3-(1-((5-cyclopropyl-1H-pyrazol-3-yl)amino)-3-methyl-1-oxobutan- |
| | 2-yl)phenyl)pyridin-2-yl)-4-morpholinobut-2-enamide; |
| 45 | (E)-N-(5-(3-(1-((5-cyclopropyl-1H-pyrazol-3-yl)amino)-1-oxopropan-2- |
| | yl)phenyl)pyridin-2-yl)-4-(3-fluoropyrrolidin-1-yl)but-2-enamide; |
| 46 | (E)-N-(5-(3-(1-((5-cyclopropyl-1H-pyrazol-3-yl)amino)-1-oxopropan-2- |
| | yl)phenyl)pyridin-2-yl)-4-((S)-3-fluoropyrrolidin-1-yl)but-2-enamide; |
| 47 | (E)-N-(5-(3-(1-((5-cyclopropyl-1H-pyrazol-3-yl)amino)-1-oxopropan-2- |
| | yl)phenyl)pyridin-2-yl)-4-((S)-3-fluoropyrrolidin-1-yl)but-2-enamide (Isomer-1 of |
| | compound-46); |
| 48 | E)-N-(5-(3-(1-((5-cyclopropyl-1H-pyrazol-3-yl)amino)-1-oxopropan-2- |
| | yl)phenyl)pyridin-2-yl)-4-((S)-3-fluoropyrrolidin-1-yl)but-2-enamide (Isomer-2 of |
| | compound-46); |
| 49 | (E)-N-(5-(3-(1-((5-cyclopropyl-1H-pyrazol-3-yl)amino)-1-oxopropan-2- |
| | yl)phenyl)pyridin-2-yl)-4-((R)-3-fluoropyrrolidin-1-yl)but-2-enamide |
| 50 | (E)-N-(5-(3-(1-((5-cyclopropyl-1H-pyrazol-3-yl)amino)-1-oxopropan-2- |
| | yl)phenyl)pyridin-2-yl)-4-((R)-3-fluoropyrrolidin-1-yl)but-2-enamide (Isomer-1 of |
| | compound-49); |
| 51 | (E)-N-(5-(3-(1-((5-cyclopropyl-1H-pyrazol-3-yl)amino)-1-oxopropan-2- |
| | yl)phenyl)pyridin-2-yl)-4-((R)-3-fluoropyrrolidin-1-yl)but-2-enamide (Isomer-2 of |
| | compound-49); |
| 52 | (E)-N-(5-(3-(1-((5-cyclopropyl-1H-pyrazol-3-yl)amino)-1-oxopropan-2- |
| | yl)phenyl)pyridin-2-yl)-4-(pyrrolidin-1-yl)but-2-enamide; |
| 53 | (E)-N-(5-(3-(1-((5-cyclopropyl-1H-pyrazol-3-yl)amino)-1-oxopropan-2-yl)- |
| | phenyl)pyridin-2-yl)-4-(pyrrolidin-1-yl)but-2-enamide (Isomer-1 of compound-52); |
| 54 | (E)-N-(5-(3-(1-((5-cyclopropyl-1H-pyrazol-3-yl)amino)-1-oxopropan-2-yl)- |
| | phenyl)pyridin-2-yl)-4-(pyrrolidin-1-yl)but-2-enamide (Isomer-2 of compound-52); |
| 55 | (E)-N-(5-(3-(1-((5-cyclopropyl-1H-pyrazol-3-yl)amino)-1-oxopropan-2- |
| | yl)phenyl)pyridin-2-yl)-4-(diethylamino)but-2-enamide; |
| 56 | (E)-N-(5-(3-(1-((5-cyclopropyl-1H-pyrazol-3-yl)amino)-3-methyl-1-oxobutan-2- |
| | yl)phenyl)pyridin-2-yl)-4-(dimethylamino)but-2-enamide; |
| 57 | (E)-N-(5-(3-(1-((5-cyclopropyl-1H-pyrazol-3-yl)amino)-1-oxopropan-2- |
| | yl)phenyl)pyridin-2-yl)-4-(3-fluoropiperidin-1-yl)but-2-enamide; |
| 58 | (E)-N-(6-(3-(1-((5-cyclopropyl-1H-pyrazol-3-yl)amino)-1-oxopropan-2-yl)phenyl)- |
| | 2-fluoropyridin-3-yl)-4-morpholinobut-2-enamide; |
| 59 | (E)-N-(5-(3-(1-((5-cyclopropyl-1H-pyrazol-3-yl)amino)-1-oxopropan-2-yl)phenyl)- |
| | 6-fluoropyridin-2-yl)-4-morpholinobut-2-enamide; |
| 60 | (E)-N-(5-(3-(1-((5-cyclopropyl-1H-pyrazol-3-yl)amino)-1-oxopropan-2- |
| | yl)phenyl)pyridin-3-yl)-4-morpholinobut-2-enamide; |
| 61 | (E)-N-(3′-(1-((5-cyclopropyl-1H-pyrazol-3-yl)amino)-1-oxopropan-2-yl)-[1,1′- |
| | biphenyl]-3-yl)-4-morpholinobut-2-enamide; |
| 62 | (E)-N-(5-(3-(1-((5-cyclopropyl-1H-pyrazol-3-yl)amino)-1-oxopropan-2- |
| | yl)phenyl)pyridin-2-yl)-4-(methoxy(methyl)amino)but-2-enamide; |
| 63 | (E)-N-(5-(3-(1-((5-cyclopropyl-1H-pyrazol-3-yl)amino)-1-oxopropan-2-yl)phenyl)- |
| | 3-fluoropyridin-2-yl)-4-morpholinobut-2-enamide; |
| 64 | (E)-N-(6-(3-(1-((5-cyclopropyl-1H-pyrazol-3-yl)amino)-1-oxopropan-2-yl)phenyl)- |
| | 2-fluoropyridin-3-yl)-4-(3-fluoropyrrolidin-1-yl)but-2-enamide; |
| 65 | N-(3′-(1-((5-cyclopropyl-1H-pyrazol-3-yl)amino)-1-oxopropan-2-yl)-3-methoxy- |
| | [1,1′-biphenyl]-4-yl)acrylamide; |
| 66 | N-(3′-(1-((5-cyclopropyl-1H-pyrazol-3-yl)amino)-1-oxopropan-2-yl)-3-methyl-[1,1′- |
| | biphenyl]-4-yl)acrylamide; |
| 67 | N-(3′-(1-((5-cyclopropyl-1H-pyrazol-3-yl)amino)-1-oxopropan-2-yl)-3,5-dimethyl- |
| | [1,1′-biphenyl]-4-yl)acrylamide; |
| 68 | (E)-N-(5-(3-(1-((5-cyclopropyl-1H-pyrazol-3-yl)amino)-1-oxopropan-2- |
| | yl)phenyl)pyridin-2-yl)-4-((2-methoxyethyl)(methyl)amino)but-2-enamide; |
| 69 | (E)-N-(5-(3-(1-((5-cyclopropyl-1H-pyrazol-3-yl)amino)-1-oxopropan-2- |
| | yl)phenyl)pyridin-2-yl)-4-(1H-imidazol-1-yl)but-2-enamide; |
| 70 | (E)-N-(5-(3-(1-((5-cyclopropyl-1H-pyrazol-3-yl)amino)-1-oxopropan-2- |
| | yl)phenyl)pyridin-2-yl)-4-((S)-2-(methoxymethyl)pyrrolidin-1-yl)but-2-enamide; |
| 71 | (E)-N-(5-(3-(1-((5-cyclopropyl-1H-pyrazol-3-yl)amino)-1-oxopropan-2- |
| | yl)phenyl)pyridin-2-yl)-4-((S)-2-(methoxymethyl)pyrrolidin-1-yl)but-2-enamide |
| | (Isomer-1 of compound-70); |
| 72 | (E)-N-(5-(3-(1-((5-cyclopropyl-1H-pyrazol-3-yl)amino)-1-oxopropan-2- |
| | yl)phenyl)pyridin-2-yl)-4-((S)-2-(methoxymethyl)pyrrolidin-1-yl)but-2-enamide |
| | (Isomer-2 of compound-70); |
| 73 | N-(3′-(1-((5-cyclopropyl-1H-pyrazol-3-yl)amino)-1-oxopropan-2-yl)-2-methoxy- |
| | [1,1′-biphenyl]-4-yl)acrylamide; |
| 74 | N-((5-(3-(1-((5-cyclopropyl-1H-pyrazol-3-yl)amino)-1-oxopropan-2- |
| | yl)phenyl)pyridin-2-yl)methyl)acrylamide; |
| 75 | N-(3′-(1-((5-cyclopropyl-1H-pyrazol-3-yl)amino)-1-oxopropan-2-yl)-2-methyl-[1,1′- |
| | biphenyl]-4-yl)acrylamide; |
| 76 | 2-(3-(1-acryloylindolin-5-yl)phenyl)-N-(5-cyclopropyl-1H-pyrazol-3- |
| | yl)propanamide; |
| 77 | (E)-N-(5-(3-(1-((5-cyclopropyl-1H-pyrazol-3-yl)amino)-1-oxopropan-2- |
| | yl)phenyl)pyridin-2-yl)-4-((2S,4S)-4-fluoro-2-(methoxymethyl)pyrrolidin-1-yl)but- |
| | 2-enamide; |
| 78 | (E)-4-(3-cyanopyrrolidin-1-yl)-N-(5-(3-(1-((5-cyclopropyl-1H-pyrazol-3-yl)amino)- |
| | 1-oxopropan-2-yl)phenyl)pyridin-2-yl)but-2-enamide; |
| 79 | N-(6-(3-(1-((5-cyclopropyl-1H-pyrazol-3-yl)amino)-1-oxopropan-2- |
| | yl)phenyl)pyrazin-2-yl)acrylamide; |
| 80 | (E)-N-(3′-(1-((5-cyclopropyl-1H-pyrazol-3-yl)amino)-1-oxopropan-2-yl)-3-fluoro- |
| | [1,1′-biphenyl]-4-yl)-4-((S)-3-fluoropyrrolidin-1-yl)but-2-enamide; |
| 81 | methyl ((E)-4-((5-(3-(1-((5-cyclopropyl-1H-pyrazol-3-yl)amino)-1-oxopropan-2- |
| | yl)phenyl)pyridin-2-yl)amino)-4-oxobut-2-en-1-yl)-L-prolinate; |
| 82 | (E)-4-((S)-2-(cyanomethyl)pyrrolidin-1-yl)-N-(5-(3-(1-((5-cyclopropyl-1H-pyrazol- |
| | 3-yl)amino)-1-oxopropan-2-yl)phenyl)pyridin-2-yl)but-2-enamide; |
| 83 | 4-acrylamido-N-(3-((5-ethyl-1H-pyrazol-3-yl)amino)phenyl)benzamide; |
| 84 | (E)-N-(5-cyclopropyl-1H-pyrazol-3-yl)-2-(3-(1-(4-(dimethylamino)but-2-enoyl)- |
| | 1,2,3,6-tetrahydropyridin-4-yl)phenyl)propanamide; |
| 85 | (E)-N-(5-cyclopropyl-1H-pyrazol-3-yl)-2-(3-(1-(4-(dimethylamino)but-2- |
| | enoyl)piperidin-4-yl)phenyl)propanamide; |
| 86 | N-(3′-(2-((5-methyl-1H-pyrazol-3-yl)amino)-2-oxoethyl)-[1,1′-biphenyl]-4- |
| | yl)acrylamide; |
| 87 | N-(3′-(1-((5-ethyl-1H-pyrazol-3-yl)amino)-1-oxopropan-2-yl)-[1,1′-biphenyl]-4- |
| | yl)acrylamide; |
| 88 | N-(3′-(1-((5-(tert-butyl)-1H-pyrazol-3-yl)amino)-1-oxopropan-2-yl)-[1,1′-biphenyl]- |
| | 4-yl)acrylamide; |
| 89 | (E)-N-(3-((1H-indazol-3-yl)amino)phenyl)-4-(4-(dimethylamino)but-2- |
| | enamido)benzamide; |
| 90 | N-(3′-(1-((1H-indazol-3-yl)amino)-1-oxopropan-2-yl)-[1,1′-biphenyl]-4- |
| | yl)acrylamide; |
| 91 | (E)-N-(3′-(1-((1H-indazol-3-yl)amino)-1-oxopropan-2-yl)-[1,1′-biphenyl]-4-yl)-4- |
| | (dimethylamino)but-2-enamide; |
| 92 | (E)-N-(6-(3-(1-((5-cyclopropyl-1H-pyrazol-3-yl)amino)-1-oxopropan-2- |
| | yl)phenyl)pyrazin-2-yl)-4-(pyrrolidin-1-yl)but-2-enamide; |
| 93 | (S,E)-N-(5-(3-(1-((5-cyclopropyl-1H-pyrazol-3-yl)amino)-1-oxopropan-2- |
| | yl)phenyl)pyridin-2-yl)-4-morpholinobut-2-enamide; |
| 94 | (E)-N-(5-cyclopropyl-1H-pyrazol-3-yl)-1-(3-(6-(4-(pyrrolidin-1-yl)but-2- |
| | enamido)pyridin-3-yl)phenyl)cyclopropane-1-carboxamide; |
| 95 | (E)-N-(5-(3-(1-((5-cyclopropyl-1H-pyrazol-3-yl)amino)-2-methyl-1-oxopropan-2- |
| | yl)phenyl)pyridin-2-yl)-4-(pyrrolidin-1-yl)but-2-enamide; |
| 96 | (E)-N-(5-(3-(1-((5-cyclopropyl-1H-pyrazol-3-yl)amino)-3-methyl-1-oxobutan-2- |
| | yl)phenyl)pyridin-2-yl)-4-(pyrrolidin-1-yl)but-2-enamide; |
| 97 | (E)-N-(5-(3-(1-((5-cyclopropyl-1H-pyrazol-3-yl)amino)-3-methyl-1-oxobutan-2- |
| | yl)phenyl)pyridin-2-yl)-4-(3-fluoropyrrolidin-1-yl)but-2-enamide; |
| 98 | (E)-N-(5-(3-(1-((5-cyclopropyl-1H-pyrazol-3-yl)amino)-3-methyl-1-oxobutan-2- |
| | yl)phenyl)pyridin-2-yl)-4-(3-hydroxypyrrolidin-1-yl)but-2-enamide; |
| 99 | (E)-N-(5-(3-(1-((5-cyclopropyl-1H-pyrazol-3-yl)amino)-3-methyl-1-oxobutan-2- |
| | yl)phenyl)pyridin-2-yl)-4-(piperidin-1-yl)but-2-enamide; |
| 100 | (E)-N-(5-(3-(1-((5-cyclopropyl-1H-pyrazol-3-yl)amino)-3-methyl-1-oxobutan-2- |
| | yl)phenyl)pyridin-2-yl)-4-(3-fluoropiperidin-1-yl)but-2-enamide; |
| 101 | (E)-N-(5-(3-(1-((5-cyclopropyl-1H-pyrazol-3-yl)amino)-3-methyl-1-oxobutan-2- |
| | yl)phenyl)pyridin-2-yl)-4-(3-hydroxypiperidin-1-yl)but-2-enamide; |
| 102 | (E)-N-(5-(3-(1-((5-cyclopropyl-1H-pyrazol-3-yl)amino)-3-methyl-1-oxobutan-2- |
| | yl)phenyl)pyridin-2-yl)-4-(4-fluoropiperidin-1-yl)but-2-enamide; |
| 103 | (E)-N-(5-(3-(1-((5-cyclopropyl-1H-pyrazol-3-yl)amino)-3-methyl-1-oxobutan-2- |
| | yl)phenyl)pyridin-2-yl)-4-(4-hydroxy-4-methylpiperidin-1-yl)but-2-enamide; |
| 104 | (E)-N-(5-(3-(1-((5-cyclopropyl-1H-pyrazol-3-yl)amino)-3-methyl-1-oxobutan-2- |
| | yl)phenyl)pyridin-2-yl)-4-(4,4-difluoropiperidin-1-yl)but-2-enamide; |
| 105 | (E)-N-(5-(3-(1-((5-cyclopropyl-1H-pyrazol-3-yl)amino)-3-methyl-1-oxobutan-2- |
| | yl)phenyl)pyridin-2-yl)-4-(diethylamino)but-2-enamide; |
| 106 | (E)-N-(1-(3-(1-((5-cyclopropyl-1H-pyrazol-3-yl)amino)-1-oxopropan-2- |
| | yl)phenyl)piperidin-4-yl)-4-(dimethylamino)but-2-enamide; |
| 107 | (E)-N-(3-(3-(1-((5-cyclopropyl-1H-pyrazol-3-yl)amino)-1-oxopropan-2-yl)phenyl)- |
| | 1-methyl-1H-pyrazol-5-yl)-4-(dimethylamino)but-2-enamide; |
| 108 | (E)-N-(5-cyclopropyl-1H-pyrazol-3-yl)-2-(3-(4-(4-(dimethylamino)but-2- |
| | enoyl)piperazin-1-yl)phenyl)propanamide; |
| 109 | 3-(1-((5-cyclopropyl-1H-pyrazol-3-yl)amino)-1-oxopropan-2-yl)phenyl (E)-4-(4- |
| | (dimethylamino)but-2-enoyl)piperazine-1-carboxylate; |
| 110 | (E)-4-((S)-2-cyanopyrrolidin-1-yl)-N-(5-(3-((S)-1-((5-cyclopropyl-1H-pyrazol-3- |
| | yl)amino)-1-oxopropan-2-yl)phenyl)pyridin-2-yl)but-2-enamide; |
| 111 | (E)-4-((2S,4S)-2-(cyanomethyl)-4-fluoropyrrolidin-1-yl)-N-(5-(3-((S)-1-((5- |
| | cyclopropyl-1H-pyrazol-3-yl)amino)-1-oxopropan-2-yl)phenyl)pyridin-2-yl)but-2- |
| | enamide; |
| 112 | (E)-N-(3′-((S)-1-((5-cyclopropyl-1H-pyrazol-3-yl)amino)-1-oxopropan-2-yl)-3- |
| | fluoro-[1,1′-biphenyl]-4-yl)-4-((S)-2-(methoxymethyl)pyrrolidin-1-yl)but-2- |
| | enamide; |
| 113 | (E)-N-(3-(1-((5-cyclopropyl-1H-pyrazol-3-yl)amino)-1-oxopropan-2-yl)phenyl)-4- |
| | (4-(dimethylamino)but-2-enoyl)morpholine-2-carboxamide; |
| 114 | (E)-N-(3-(1-((5-cyclopropyl-1H-pyrazol-3-yl)amino)-1-oxopropan-2-yl)phenyl)-4- |
| | (4-(dimethylamino)but-2-enamido)tetrahydro-2H-pyran-3-carboxamide; |
| 115 | (E)-N-(3-(1-((5-cyclopropyl-1H-pyrazol-3-yl)amino)-1-oxopropan-2-yl)phenyl)-1- |
| | (4-(dimethylamino)but-2-enoyl)pyrrolidine-2-carboxamide; |
| 116 | (E)-N-(3-(1-((5-cyclopropyl-1H-pyrazol-3-yl)amino)-1-oxopropan-2-yl)phenyl)-1- |
| | (4-(dimethylamino)but-2-enoyl)piperidine-2-carboxamide; |
| 117 | (E)-N-(3-(1-((5-cyclopropyl-1H-pyrazol-3-yl)amino)-1-oxopropan-2-yl)phenyl)-1- |
| | (4-(dimethylamino)but-2-enoyl)piperidine-3-carboxamide; |
| 118 | (E)-N-(3-(1-((5-cyclopropyl-1H-pyrazol-3-yl)amino)-1-oxopropan-2-yl)phenyl)-4- |
| | (dimethylamino)but-2-enamide; |
| 119 | (E)-N-(4-(1-((5-cyclopropyl-1H-pyrazol-3-yl)amino)-1-oxopropan-2-yl)phenyl)-4- |
| | (dimethylamino)but-2-enamide; |
| 120 | (E)-N-(2-(1-((5-cyclopropyl-1H-pyrazol-3-yl)amino)-1-oxopropan-2-yl)phenyl)-4- |
| | (dimethylamino)but-2-enamide; |
| 121 | 2-(1-((5-cyclopropyl-1H-pyrazol-3-yl)amino)-1-oxopropan-2-yl)phenyl (E)-4-(4- |
| | (dimethylamino)but-2-enoyl)piperazine-1-carboxylate; |
| 122 | 4-(1-((5-cyclopropyl-1H-pyrazol-3-yl)amino)-1-oxopropan-2-yl)phenyl (E)-4-(4- |
| | (dimethylamino)but-2-enoyl)piperazine-1-carboxylate; |
| 123 | (E)-N-(5-(3-(1-((5-cyclopropyl-1H-pyrazol-3-yl)amino)-1-oxopropan-2-yl)phenyl)- |
| | 6-fluoropyridin-2-yl)-4-(dimethylamino)but-2-enamide; |
| 124 | (E)-N-(3-(3-(1-((5-cyclopropyl-1H-pyrazol-3-yl)amino)-1-oxopropan-2- |
| | yl)phenyl)pyridin-4-yl)-4-morpholinobut-2-enamide; |
| 125 | (E)-N-(3′-(1-((5-cyclopropyl-1H-pyrazol-3-yl)amino)-1-oxopropan-2-yl)-[1,1′- |
| | biphenyl]-2-yl)-4-morpholinobut-2-enamide; |
| 126 | (E)-N-(3-(1-((5-cyclopropyl-1H-pyrazol-3-yl)amino)-1-oxopropan-2-yl)phenyl)-4- |
| | (4-(dimethylamino)but-2-enoyl)piperazine-1-carboxamide; |
| 127 | (E)-N-(6-(3-((S)-1-((5-cyclopropyl-1H-pyrazol-3-yl)amino)-1-oxopropan-2- |
| | yl)phenyl)pyridin-2-yl)-4-((S)-3-fluoropyrrolidin-1-yl)but-2-enamide; |
| 128 | (E)-N-(6-(3-((S)-1-((5-cyclopropyl-1H-pyrazol-3-yl)amino)-1-oxopropan-2- |
| | yl)phenyl)pyrazin-2-yl)-4-((S)-3-fluoropyrrolidin-1-yl)but-2-enamide; |
| 129 | (E)-N-(5-(3-((S)-1-((5-cyclopropyl-1H-pyrazol-3-yl)amino)-1-oxopropan-2- |
| | yl)phenyl)pyridin-2-yl)-4-((S)-3-(trifluoromethyl)pyrrolidin-1-yl)but-2-enamide; |
| 130 | (E)-4-((2S,4S)-2-cyano-4-fluoropyrrolidin-1-yl)-N-(5-(3-((S)-1-((5-cyclopropyl-1H- |
| | pyrazol-3-yl)amino)-1-oxopropan-2-yl)phenyl)pyridin-2-yl)but-2-enamide; |
| 131 | (E)-4-((S)-2-cyanopyrrolidin-1-yl)-N-(5-(3-((S)-1-((5-cyclopropyl-1H-pyrazol-3- |
| | yl)amino)-1-oxopropan-2-yl)phenyl)pyridin-2-yl)but-2-enamide; |
| 132 | (S,E)-N-(3′-(1-((5-cyclopropyl-1H-pyrazol-3-yl)amino)-1-oxopropan-2-yl)-3-fluoro- |
| | [1,1′-biphenyl]-4-yl)-4-(diethylamino)but-2-enamide; |
| 133 | (E)-N-(4-(3-((5-cyclopropyl-1H-pyrazol-3-yl)amino)-4-methylisoquinolin-6- |
| | yl)phenyl)-4-(dimethylamino)but-2-enamide; |
| 134 | (E)-N-(4-(4-(1-((5-cyclopropyl-1H-pyrazol-3-yl)amino)-1-oxopropan-2-yl)pyridin- |
| | 2-yl)phenyl)-4-(dimethylamino)but-2-enamide; |
| 135 | (E)-N-(4-(3-(1-((5-cyclopropyl-1H-pyrazol-3-yl)amino)-1-oxopropan-2- |
| | yl)phenyl)cyclohexyl)-4-(dimethylamino)but-2-enamide; |
| 136 | (E)-N-(3-(3-(1-((5-cyclopropyl-1H-pyrazol-3-yl)amino)-1-oxopropan-2- |
| | yl)phenyl)cyclohexyl)-4-(dimethylamino)but-2-enamide; |
| 137 | (E)-4-(dimethylamino)-N-(3′-(1-oxo-1-(pyrazolo[1,5-a]pyridin-2-ylamino)propan-2- |
| | yl)-[1,1′-biphenyl]-4-yl)but-2-enamide; |
| 138 | (E)-N-(3′-(3-((5-cyclopropyl-1H-pyrazol-3-yl)amino)-1,1,1-trifluoro-3-oxopropan- |
| | 2-yl)-[1,1′-biphenyl]-4-yl)-4-(dimethylamino)but-2-enamide; |
| 139 | (E)-N-(3′-(1-((5-cyclopentyl-1H-pyrazol-3-yl)amino)-1-oxopropan-2-yl)-[1,1′- |
| | biphenyl]-4-yl)-4-(dimethylamino)but-2-enamide; |
| 140 | (E)-N-(3′-(1-((5-cyclohexyl-1H-pyrazol-3-yl)amino)-1-oxopropan-2-yl)-[1,1′- |
| | biphenyl]-4-yl)-4-(dimethylamino)but-2-enamide; |
| 141 | (E)-N-(3′-(1-((5-cyclopropyl-4-methyl-1H-pyrazol-3-yl)amino)-1-oxopropan-2-yl)- |
| | [1,1′-biphenyl]-4-yl)-4-(dimethylamino)but-2-enamide; |
| 142 | (E)-N-(3′-(1-((5-cyclopropyl-4-(pyridin-2-yl)-1H-pyrazol-3-yl)amino)-1-oxopropan- |
| | 2-yl)-[1,1′-biphenyl]-4-yl)-4-(dimethylamino)but-2-enamide; and |
| 143 | (E)-N-(3′-(3-(5-cyclopropyl-1H-pyrazol-3-yl)-1-methylureido)-[1,1′-biphenyl]-4- |
| | yl)-4-(dimethylamino)but-2-enamide; |

or a pharmaceutically acceptable salt or a stereoisomer thereof.

In some embodiments, the present methods include administering a compound of formula (I) selected from:
(E)-N-(5-(3-(1-((5-cyclopropyl-1H-pyrazol-3-yl)amino)-1-oxopropan-2-yl)phenyl)pyridin-2-yl)-4-((S)-3-fluoropyrrolidin-1-yl)but-2-enamide;
N-(5-(3-(1-((5-cyclopropyl-1H-pyrazol-3-yl)amino)-1-oxopropan-2-yl)phenyl)pyridin-2-yl)acrylamide;
(E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide;
(E)-N-(5-(3-(1-((5-cyclopropyl-1H-pyrazol-3-yl)amino)-1-oxopropan-2-yl)phenyl)pyridin-2-yl)-4-morpholinobut-2-enamide;
(E)-N-(5-(3-(1-((5-cyclopropyl-1H-pyrazol-3-yl)amino)-1-oxopropan-2-yl)phenyl)pyridin-2-yl)-4-(pyrrolidin-1-yl)but-2-enamide;
(E)-N-(5-(3-(1-((5-cyclopropyl-1H-pyrazol-3-yl)amino)-3-methyl-1-oxobutan-2-yl)phenyl)pyridin-2-yl)-4-(dimethylamino)but-2-enamide;
(E)-N-(5-(3-(1-((5-cyclopropyl-1H-pyrazol-3-yl)amino)-1-oxopropan-2-yl)phenyl)pyridin-2-yl)-4-(piperidin-1-yl)but-2-enamide;
(E)-N-(3′-(14(5-cyclopropyl-1H-pyrazol-3-yl)amino)-3-methyl-1-oxobutan-2-yl)-3-fluoro-[1,1′-biphenyl]-4-yl)-4-(dimethylamino)but-2-enamide; and
(E)-N-(5-(3-(1-((5-cyclopropyl-1H-pyrazol-3-yl)amino)-1-oxopropan-2-yl)phenyl)pyridin-2-yl)-4-(3-fluoropiperidin-1-yl)but-2-enamide.

In some embodiments, the present methods include administering a compound of formula (I) selected from:
(E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide;
(E)-N-(5-(3-(1-((5-cyclopropyl-1H-pyrazol-3-yl)amino)-1-oxopropan-2-yl)phenyl)pyridin-2-yl)-4-(pyrrolidin-1-yl)but-2-enamide; and
(E)-N-(5-(3-(1-((5-cyclopropyl-1H-pyrazol-3-yl)amino)-1-oxopropan-2-yl)phenyl)pyridin-2-yl)-4-morpholinobut-2-enamide.

In some embodiments, the compound of formula (I) is (E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide.

In some embodiments, the compound of formula (I) is (S)-(E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide.

In some embodiments, the present methods include administering a compound of formula (I) which is Compound 44A in combination with an anti-microtubule agent. In some embodiments, the anti-microtubule agent is docetaxel.

According to the preceding embodiment, Compound 44A is the (S)-(E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide.

In some embodiments, the present methods include administering a compound of formula (I) conjointly with an anti-microtubule agent.

In some embodiments, the anti-microtubule agent is selected from docetaxel, paclitaxel, vincristine, vinblastine and vindesine, or a derivative or a prodrug thereof.

According to the preceding embodiment, the anti-microtubule agent is docetaxel or a derivative or a prodrug thereof.

According to the preceding embodiment, the anti-microtubule agent is docetaxel.

In certain embodiments, the present invention provides a pharmaceutical composition comprising docetaxel, or a derivative or prodrug thereof, a compound of formula (I) or a pharmaceutically acceptable salt or a stereoisomer thereof, and a pharmaceutically acceptable carrier, diluent or excipient.

### Pharmaceutical Compositions

In certain embodiments, the present methods include a pharmaceutical composition comprising the compound as disclosed herein, optionally admixed with a pharmaceutically acceptable carrier or diluent.

As used herein, the term “composition” is intended to encompass a product comprising the specified ingredients in the specified amounts, as well as any product which results, directly or indirectly, from combination of the specified ingredients in the specified amounts.

As used herein, the term “pharmaceutical composition” refers to a composition(s) containing a therapeutically effective amount of at least one compound of formula (I) or its pharmaceutically acceptable salt; and a conventional pharmaceutically acceptable carrier.

The pharmaceutical composition(s) of the present invention can be administered orally, for example in the form of tablets, coated tablets, pills, capsules, granules or elixirs. Administration, however, can also be carried out rectally, for example in the form of suppositories, or parenterally, for example intravenously, intramuscularly or subcutaneously, in the form of injectable sterile solutions or suspensions, or topically, for example in the form of ointments or creams or transdermals, in the form of patches, or in other ways, for example in the form of aerosols or nasal sprays.

The pharmaceutical composition(s) usually contain(s) about 1% to 99%, for example, about 5% to 75%, or from about 10% to about 30% by weight of the compound of formula (I) or pharmaceutically acceptable salts thereof. The amount of the compound of formula (I) or pharmaceutically acceptable salts thereof in the pharmaceutical composition(s) can range from about 1 mg to about 1000 mg or from about 2.5 mg to about 500 mg or from about 5 mg to about 250 mg or in any range falling within the broader range of 1 mg to 1000 mg or higher or lower than the afore mentioned range.

The present invention also provides methods for formulating the disclosed compounds as for pharmaceutical administration.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters.

In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation of pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

In certain embodiments, the pharmaceutical composition comprises the active ingredient in an amount of at least about 50% by weight of the composition. In certain embodiments, the pharmaceutical composition comprises the active ingredient in an amount of at least about 60% by weight of the composition. In certain embodiments, the pharmaceutical composition comprises the active ingredient in an amount of at least about 70% by weight of the composition. In certain embodiments, the pharmaceutical composition comprises the active ingredient in an amount of at least about 80% by weight of the composition. In certain embodiments, the pharmaceutical composition comprises the active ingredient in an amount of at least about 90% by weight of the composition.

In certain embodiments, the oral pharmaceutical composition comprising the active ingredient of the present invention is a unit dose composition. In certain embodiments, the pharmaceutical composition contains about 1 mg to about 5000 mg of the active ingredient. In certain embodiments, the pharmaceutical composition contains about 100 mg to about 3000 mg of the active ingredient. In certain embodiments, the pharmaceutical composition contains about 200 mg to about 2000 mg of the active ingredient.

In certain embodiments, the active ingredient is a compound of formula (I).

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, about 0.1 to about 99.5% (more preferably, about 0.5 to about 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

The compounds of the present invention may be administered in combination with one or more other drugs (1) to complement and/or enhance prevention and/or therapeutic efficacy of the preventive and/or therapeutic drug effect of the compound of the present invention, (2) to modulate pharmacodynamics, improve absorption improvement, or reduce dosage reduction of the preventive and/or therapeutic compound of the present invention, and/or (3) to reduce or ameliorate the side effects of the preventive and/or therapeutic compound of the present invention. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds. The respective compounds may be administered by the same or different route and the same or different method.

A concomitant medicine comprising the compounds of the present invention and other drug may be administered as a combination preparation in which both components are contained in a single formulation or administered as separate formulations. The administration by separate formulations includes simultaneous administration and or administration of the formulations separated by some time intervals. In the case of the administration with some time intervals, the compound of the present invention can be administered first, followed by another drug or another drug can be administered first, followed by the compound of the present invention, so long as the two compounds are simultaneously active in the patient at least some of the time during the conjoint therapy. The administration method of the respective drugs may be administered by the same or different route and the same or different method.

The dosage of the other drug can be properly selected, based on a dosage that has been clinically used, or may be a reduced dosage that is effective when administered in combination with a compound of the present invention. The compounding ratio of the compound of the present invention and the other drug can be properly selected according to age and weight of a subject to be administered, administration method, administration time, disorder to be treated, symptom and combination thereof. For example, the other drug may be used in an amount of about 0.01 to about 100 parts by mass, based on 1 part by mass of the compound of the present invention. The other drug may be a combination of two or more kind of arbitrary drugs in a proper proportion. The other drug that complements and/or enhances the preventive and/or therapeutic efficacy of the compound of the present invention includes not only those that have already been discovered, but those that will be discovered in future, based on the above mechanism.

In certain embodiments, a compound of the invention may be conjointly administered with non-chemical methods of cancer treatment. In certain embodiments, a compound of the invention may be conjointly administered with radiation therapy. In certain embodiments, a compound of the invention may be conjointly administered with surgery, with thermoablation, with focused ultrasound therapy, with cryotherapy, or with any combination of these.

### Methods of Treatment

Disclosed herein are methods for treating or preventing cancer that comprise administering a disclosed compound with an anti-microtubule agent. In some embodiments, the cancer is prostate cancer. In some embodiments, the prostate cancer is metastatic castration-resistant prostate cancer. In some embodiments, the anti-microtubule agent is docetaxel.

In some embodiments, the present invention provides methods for treating a prostate cancer selected from the group consisting of androgen-dependent prostate cancer and androgen-independent prostate cancer. In some embodiments, the present invention provides methods wherein the prostate cancer is metastatic.

Breast cancer is cancer that develops from breast tissue. About 5-10% of cases are the result of a genetic predisposition inherited from a person's parents, including BRCA1 and BRCA2 among others. Outcomes for breast cancer vary depending on the cancer type, the extent of disease, and the person's age.

Head and neck cancer is a term used to define cancer that develops in the mouth, throat, larynx, nose, salivary glands, oral cancers or other areas of the head and neck. Most of these cancers are squamous cell carcinomas, or cancers that begin in the lining of the mouth, nose and throat. Tobacco use, heavy alcohol use, and infection with human papillomavirus (HPV) increase the risk of head and neck cancers. Eighty-five percent of head and neck cancers are linked to tobacco use, and 75 percent are associated with a combination of tobacco and alcohol use.

Most (about 90% to 95%) cancers of the stomach are adenocarcinomas. A stomach cancer or gastric cancer almost always is an adenocarcinoma. These cancers develop from the cells that form the innermost lining of the stomach (the mucosa).

Non-small cell lung cancer (NSCLC) accounts for approximately 85% of all lung cancers. Histologically, NSCLC is divided into adenocarcinoma, squamous cell carcinoma (SCC) (see the image below), and large cell carcinoma.

In some embodiments, the present invention provides methods for treating cancer selected from breast cancer, head and neck cancer, squamous cell carcinoma, stomach cancer, ovarian cancer, soft-tissue sarcoma, adenocarcinoma, lung cancer and non-small-cell lung cancer.

### Non-Hodgkin's Lymphomas

The combination of a compound of formula (I) and an anti-microtubule agent can be advantageous in treating or preventing other non-Hodgkin's lymphomas, such as mantle cell lymphoma (MCL), marginal zone lymphoma (MZL), follicular lymphoma (FL), chronic lymphocytic leukemia (CLL), small lymphocytic lymphoma (SLL), CNS lymphoma, and testicular lymphoma. Non-Hodgkin's lymphomas are cancers that begin in white blood cells called lymphocytes. These cancers can affect B -cells or T-cells, with B-cell lymphomas being more prevalent. Indolent lymphomas grow slowly, such as FL and MZL Aggressive lymphomas that rapidly grow include DLBCL. MZL often occurs in the stomach. Mucosa-associated lymphoid tissue (MALT) lymphoma is the most common form of marginal zone lymphoma that occurs outside the lymph nodes. Other types include nodal marginal zone lymphoma occurring inside the lymph nodes and splenic marginal zone lymphoma within the spleen.

CLL is the most common type of leukemia. CLL affects B cell lymphocytes, which originate in the bone marrow, develop in the lymph nodes, and normally fight infection by producing antibodies. In CLL, B cells grow in an uncontrolled manner and accumulate in the bone marrow and blood, where they crowd out healthy blood cells. CLL is a stage of small lymphocytic lymphoma (SLL), a type of B -cell lymphoma, which presents primarily in the lymph nodes. CLL and SLL are considered the same underlying disease, just with different appearances. Most people are diagnosed without symptoms as the result of a routine blood test that shows a high white blood cell count. As it advances, CLL results in swollen lymph nodes, spleen, and liver, and eventually anemia and infections.

CNS lymphoma, also known as microglioma and primary brain lymphoma, is a primary intracranial tumor appearing mostly in patients with severe immunodeficiency (typically patients with AIDS). CNS lymphoma is highly associated with Epstein-Barr virus (EBV) infection (>90%) in immunodeficient patients (such as those with AIDS and those immunosuppressed). CNS lymphoma is a type of DLBCL.

Testicular lymphoma is a rare extranodal presentation of non-Hodgkin's lymphoma. Testicular lymphoma is a type of DLBCL. Testicular lymphoma is frequently associated with Burkitt's NHL and non-Burkitt's small-cell NHL, T-ALL, primary testicular diffuse large-cell lymphoma, HIV-associated aggressive B -cell lymphoma, and HTLV-1 associated lymphoma. Lymphoma is the most common secondary testicular cancer. Among men older than 50, testicular lymphoma is more common than primary testicular tumors.

### Leukemias

The combination of a compound of formula (I) and an anti-microtubule agent can be advantageous in treating or preventing leukemia including, but not limited to, acute myeloid leukemia (AML) and myelodysplastic syndromes (MDS). Leukemia is a group of cancers that usually begin in the bone marrow and result in high numbers of abnormal white blood cells. These white blood cells are not fully developed and are called blasts or leukemia cells. The exact cause of leukemia is unknown.

In acute lymphoblastic leukemia (ALL) and AML, a rapid increase in the number of immature blood cells occurs. The crowding that results from such cells makes the bone marrow unable to produce healthy blood cells. Immediate treatment is required in acute leukemia because of the rapid progression and accumulation of the malignant cells, which then spill over into the bloodstream and spread to other organs of the body. In chronic lymphocytic leukemia (CLL) and chronic myelogenous leukemia (CML), an excessive buildup of relatively mature, but still abnormal, white blood cells occurs. Typically taking months or years to progress, the cells are produced at a much higher rate than normal, resulting in many abnormal white blood cells. Whereas acute leukemia is treated immediately, chronic forms are sometimes monitored for some time before treatment to ensure maximum effectiveness of therapy. In ALL and CLL, the cancerous change takes place in a type of marrow cell that normally goes on to form lymphocytes, which are infection-fighting immune system cells. Most lymphocytic leukemias involve a specific subtype of lymphocyte, the B cell. In AML and CML, the cancerous change takes place in a type of marrow cell that normally goes on to form red blood cells, some other types of white cells, and platelets.

Myelodysplastic syndromes are a group of cancers in which immature blood cells in the bone marrow do not mature and therefore do not become healthy blood cells. Some types may develop into acute myeloid leukemia. The World Health Organization has identified the following categories of myelodysplastic syndrome: refractory anemia, refractory anemia with ringed sideroblasts, refractory anemia with excess blasts in transformation, and chronic myelomonocytic leukemia. Risk factors for developing one of these syndromes include previous chemotherapy or radiation therapy, exposure to certain chemicals such as tobacco smoke, pesticides, and benzene, and exposure to heavy metals such as mercury or lead.

Problems with blood cell formation result in some combination of low red blood cells, low platelets, and low white blood cells. Some types have an increase in immature blood cells, called blasts, in the bone marrow or blood. The types of MDS are based on specific changes in the blood cells and bone marrow. The typical survival rate following diagnosis is 2.5 years.

### Solid Malignant Tumors

Sarcomas, carcinomas, melanomas, and glioblastomas are the main types of solid malignant tumors. Sarcomas are tumors in a blood vessel, bone, fat tissue, ligament, lymph vessel, muscle or tendon. Carcinomas are tumors that form in epithelial cells. Epithelial cells are found in the skin, glands and the linings of organs. Melanomas are tumors that develop in the pigment-containing cells known as melanocytes. Glioblastoma is an aggressive cancer that begins in the brain. They can either start from normal brain cells or develop from an existing low-grade astrocytoma. A solid tumor grows in an anatomical site outside the bloodstream (in contrast, for example, to cancers of hematopoietic origin such as leukemias) and requires the formation of small blood vessels and capillaries to supply nutrients, etc. to the growing tumor mass. Non-limiting examples of solid malignant tumors include biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma, glioblastomas; medulloblastoma), cervical cancer (e.g., cervical adenocarcinoma), colorectal cancer (e.g., colon cancer, rectal cancer, colorectal adenocarcinoma), gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), pancreatic cancer (e.g., pancreatic andenocarcinoma, intraductal papillary mucinous neoplasm (IPMN)), prostate cancer (e.g., prostate adenocarcinoma), skin cancer (e.g., squamous cell carcinoma (SCC), keratoacanthoma, melanoma, basal cell carcinoma (BCC)) and soft tissue sarcoma (e.g., malignant fibrous histiocytoma (MFH), liposarcoma, malignant peripheral nerve sheath tumor (MPNST), chondrosarcoma, fibrosarcoma, myxosarcoma, osteosarcoma).

### Additional Malignancies

In addition to the cancers discussed above, the disclosed methods in this application are useful in the treatment of a wide variety of cancers.

In certain embodiments, the cancer is Acute Lymphoblastic Leukemia (ALL), Acute Myeloid Leukemia (AML), Adrenocortical Carcinoma, Anal Cancer, Appendix Cancer, Atypical Teratoid/Rhabdoid Tumor, Basal Cell Carcinoma, Bile Duct Cancer, Bladder Cancer, Bone Cancer, Brain Tumor, Astrocytoma, Brain and Spinal Cord Tumor, Brain Stem Glioma, Central Nervous System Atypical Teratoid/Rhabdoid Tumor, Central Nervous System Embryonal Tumors, Breast Cancer, Bronchial Tumors, Burkitt Lymphoma, Carcinoid Tumor, Carcinoma of Unknown Primary, Central Nervous System Cancer, Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma, Ductal Carcinoma In Situ (DCIS), Embryonal Tumors, Endometrial Cancer, Ependymoblastoma, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, fibrosarcoma, Fibrous Histiocytoma of Bone, Gallbladder Cancer, Gastric Cancer, Gastrointestinal Carcinoid Tumor, Gastrointestinal Stromal Tumors (GIST), Germ Cell Tumor, Ovarian Germ Cell Tumor, Gestational Trophoblastic Tumor, Glioma, Hairy Cell Leukemia, Head and Neck Cancer, Heart Cancer, Hepatocellular Cancer, Histiocytosis, Langerhans Cell Cancer, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer, Lobular Carcinoma In Situ (LCIS), Lung Cancer, Lymphoma, AIDS-Related Lymphoma, Macroglobulinemia, Male Breast Cancer, Medulloblastoma, Medulloepithelioma, Melanoma, Merkel Cell Carcinoma, Malignant Mesothelioma, Metastatic Squamous Neck Cancer with Occult Primary, Midline Tract Carcinoma Involving NUT Gene, Mouth Cancer, Multiple Endocrine Neoplasia Syndrome, Multiple Myeloma/Plasma Cell Neoplasm, Mycosis Fungoides, Myelodysplastic Syndrome, Myelodysplastic/Myeloproliferative Neoplasm, Chronic Myelogenous Leukemia (CML), Acute Myeloid Leukemia (AML), Myeloma, Multiple Myeloma, Chronic Myeloproliferative Disorder, Nasal Cavity Cancer, Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip Cancer, Oropharyngeal Cancer, Osteosarcoma, Ovarian Cancer, Pancreatic Cancer, Papillomatosis, Paraganglioma, Paranasal Sinus Cancer, Nasal Cavity Cancer, Parathyroid Cancer, Penile Cancer, Pharyngeal Cancer, Pheochromocytoma, Pineal Parenchymal Tumors of Intermediate Differentiation, Pineoblastoma, Pituitary Tumor, Plasma Cell Neoplasm, Pleuropulmonary Blastoma, Breast Cancer, Primary Central Nervous System (CNS) Lymphoma, Prostate Cancer, Rectal Cancer, Renal Cell Cancer, Clear cell renal cell carcinoma, Renal Pelvis Cancer, Ureter Cancer, Transitional Cell Cancer, Retinoblastoma, Rhabdomyosarcoma, Salivary Gland Cancer, Sarcoma, Sézary Syndrome, Skin Cancer, Small Cell Lung Cancer, Small Intestine Cancer, Soft Tissue Sarcoma, Squamous Cell Carcinoma, Squamous Neck Cancer with Occult Primary, Squamous Cell Carcinoma of the Head and Neck (HNSCC), Stomach Cancer, Supratentorial Primitive Neuroectodermal Tumors, T-Cell Lymphoma, Testicular Cancer, Throat Cancer, Thymoma, Thymic Carcinoma, Thyroid Cancer, Transitional Cell Cancer of the Renal Pelvis and Ureter, Triple Negative Breast Cancer (TNBC), Gestational Trophoblastic Tumor, Unknown Primary, Unusual Cancer of Childhood, Urethral Cancer, Uterine Cancer, Uterine Sarcoma, Waldenström Macroglobulinemia, or Wilms Tumor.

In certain embodiments, the cancer is carcinomas of the breast, liver, lung, colon, kidney, bladder, small cell lung cancer, non-small cell lung cancer, head and neck, thyroid, esophagus, stomach, pancreas, ovary, gall bladder, cervix, prostate, skin, squamous cell carcinoma, hematopoietic tumors of lymphoid lineage, leukemia, acute lymphoblastic leukemia, acute lymphocytic leukemia, Hodgkin's lymphoma, non-Hodgkin's lymphoma, B-cell lymphoma, T-cell lymphoma, hairy cell lymphoma, myeloma, mantle cell lymphoma, Burkett's lymphoma, hematopoietic tumors of myeloid lineage, acute and chronic myelogenous leukemias, myelodysplastic syndrome, promyelocytic leukemia, tumors of mesenchymal origin, fibrosarcoma, rhabdomyosarcoma, tumors of the central and peripheral nervous system, astrocytoma, neuroblastoma, glioma, schwannomas, seminoma, melanoma, osteosarcoma, teratocarcinoma, keratoacanthoma, xeroderma pigmentosum, thyroid follicular cancer and Kaposi's sarcoma.

In further embodiments of the invention, the cancer is selected from bladder cancer, breast cancer, esophageal cancer, gastric cancer, head & neck cancer, Kaposi's sarcoma, lung cancer (including non-small cell lung cancer and small cell lung cancer), melanoma, ovarian cancer, pancreatic cancer, penile cancer, prostate cancer, testicular germcell cancer, thymoma and thymic carcinoma.

Disclosed herein are methods for treating or preventing cancer that comprise administering a compound of formula (I) with an anti-microtubule agent.

Disclosed herein are methods for treating or preventing prostate cancer that comprise administering a compound of formula (I) with an anti-microtubule agent. Also disclosed herein are methods for treating or preventing metastatic castration-resistant prostate cancer.

In some embodiments, conjointly administering the anti-microtubule agent with a compound of formula (I) provides improved efficacy relative to separately administering the compound of formula (I), and the anti-microtubule agent. In some embodiments, conjointly administering the anti-microtubule agent with a compound of formula (I) provides a synergistic effect.

In some embodiments, the compound of formula (I) and the anti-microtubule agent are administered simultaneously or sequentially. In other embodiments, the anti-microtubule agent is administered within about 5 minutes to within about 168 hours prior to or after administration of the compound of formula (I).

In some embodiments, the anti-microtubule agent is docetaxel.

Disclosed herein are methods for treating or preventing cancer that comprise administering a compound of formula (I) with docetaxel.

Disclosed herein are methods for treating or preventing prostate cancer that comprise administering a compound of formula (I) with docetaxel. Also disclosed herein are methods for treating or preventing metastatic castration-resistant prostate cancer.

In some embodiments, conjointly administering docetaxel with a compound of formula (I) provides improved efficacy relative to separately administering the compound of formula (I), and docetaxel. In some embodiments, conjointly administering docetaxel with a compound of formula (I) provides a synergistic effect.

In some embodiments, the compound of formula (I) and docetaxel are administered simultaneously or sequentially. In other embodiments, docetaxel is administered within about minutes to within about 168 hours prior to or after administration of the compound of formula (I).

In certain embodiments, the present invention relates to a compound, or a pharmaceutically acceptable salt thereof as disclosed herein, for use in treating or preventing cancer conjointly with an anti-microtubule agent as discussed herein. In certain embodiments, the present invention relates to use of a compound of formula (I) or a pharmaceutically acceptable salt thereof as disclosed herein, for the preparation of a medicament for treating or preventing cancer conjointly with an anti-microtubule agent as discussed herein.

In certain preceding embodiments, the anti-microtubule agent is docetaxel.

Compounds suitable for the compositions and methods disclosed herein are disclosed in WO2016/193939 A1, which is incorporated by reference in its entirety, and in particular for the compounds disclosed therein as CDK7 inhibitors.

### METHODS/EXPERIMENTAL PROCEDURE

### Synthesis of Compound-44A & 44B

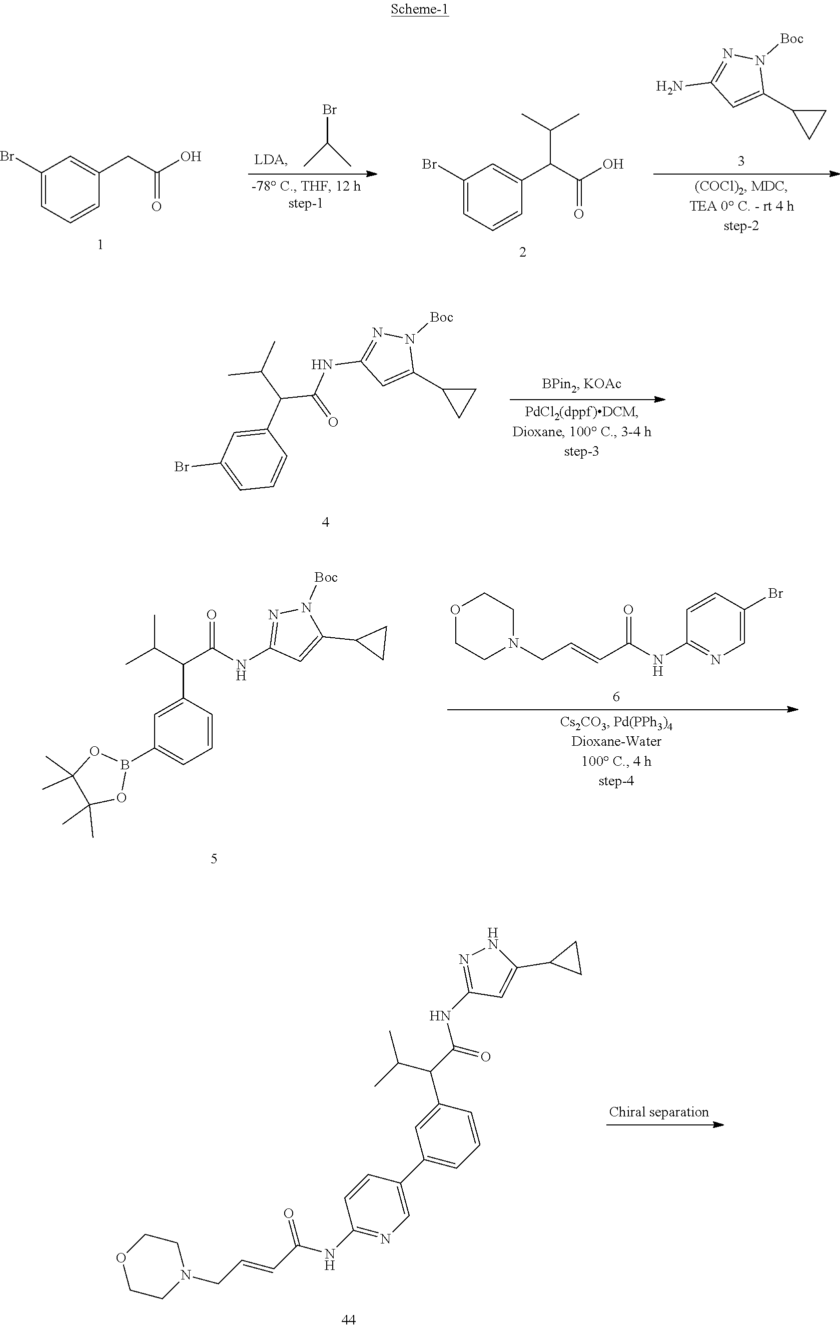

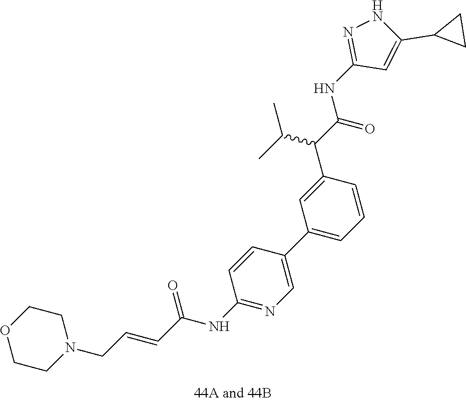 [SMILES: CC(C)C(C(=O)Nc1cc(C2CC2)[nH]n1)c1cccc(-c2ccc(NC(=O)/C=C/CN3CCOCC3)nc2)c1]

### Step-1: Synthesis of 2-(3-bromophenyl)-3-methylbutanoic Acid

2M LDA (698 mL, 1.38 mol) was added to a solution of 2-(3-bromophenyl) acetic acid (reagent-1, 150 g, 0.69 mol) in THF (700 mL) at −78° C. over a period of 30 min. The reaction mass was stirred for 2 h at −78° C. followed by the drop wise addition of Isopropyl bromide (255 g, 2.07 mol) over a period of 30 min at −78° C. The reaction mass was stirred at room temperature for overnight. The reaction mass was quenched with 1N HCl (pH 2) and product extracted to ethyl acetate (500 mL×3). The combined organic layer washed with water followed by brine, dried and concentrated under reduced pressure to afford the title crude compound which was purified by silica column by eluting with 0-10% ethyl acetate-hexane system to afford the title compound 2 (150 g, 83% yield). LCMS: m/z=254.80 (M-2H)⁻

### Step-2: Synthesis of tert-butyl 3-(2-(3-bromophenyl)-3-methylbutanamido)-5-cyclopropyl-1H-pyrazole-1-carboxylate

2-(3-bromophenyl)-3-methylbutanoic acid (intermediate-2, 70 g, 0.0.27 mol) was dissolved in dry DCM (500 mL) and added oxalyl chloride (68 mL, 0.78 mol) dropwise at 0° C. followed by addition of catalytic amount of DMF (0.8 mL) and maintained reaction mass at same temperature for 30 min. The reaction mass was allowed to room temperature and stirred for 4 h, distilled off the solvent and excess oxalyl chloride under vacuum. Re-dissolved the residue in DCM (250 mL) and added slowly to the cooled solution of tert-butyl 3-amino-5-cyclopropyl-1H-pyrazole-1-carboxylate (intermediate-3, 49g, 0.218 mol) and TEA (55 mL, 0.546 mol) in THF (250 mL) at 0° C. for 30 min, The reaction was stirred at room temperature for 12 h then the reaction mass was concentrated under reduced pressure and the residue was dissolved in DCM, washed with saturated NaHCO3 solution and brine. The organic layer was dried over anhydrous sodium sulphate and concentrated under reduced pressure, the crude was purified by silica gel column chromatography by eluting with 15% ethyl acetate-hexane to afford the title compound 4 (90g, 71%) LCMS: m/z=363.80 (M-Boc+2).

### Step-3: Synthesis of tert-butyl 5-cyclopropyl-3-(3-methyl-2-(3-(4,4,5,5-tetramethyl-1,3,2-dioxaborolan-2-yl)phenyl)butanamido)-1H-pyrazole-1-carboxylate

To a degas sed solution of tert-butyl 3-(2-(3-bromophenyl)-3-methylbutanamido)-5-cyclopropyl-1H-pyrazole-1-carboxylate (intermediate-4, 90 g, 0.193 mol) and 4,4,4′,4′,5,5,5′,5′-octamethyl-2,2′-bi(1,3,2-dioxaborolane) (62 g, 0.251 mol) in 1,4-Dioxane (500 mL) was added potassium acetate (37.80 g, 0.386 mol). The reaction mass was allowed to stir for 10 min with degassing at RT and added PdCl2 (dppf). DCM complex (12.5 g, 0.015 mol). The reaction mass was heated for 3-4 h at 100° C. Reaction mixture cooled to RT and filtered on celite bed, filtrate evaporated to get dark brown liquid. The crude material was purified by silica column chromatography by eluting with 20% ethyl acetate in hexane to afford the compound 5 (90 g, 86%). LCMS: m/z=410 (M-Boc+1)+.

### Step-4: Synthesis of (E)-N-(5-(3-(14(5-cyclopropyl-1H-pyrazol-3-yl)amino)-3-methyl-1-oxobutan-2-yl)phenyl)pyridin-2-yl)-4-morpholinobut-2-enamide

To a degassed solution of tert-butyl 5-cyclopropyl-3-(3-methyl-2-(3-(4,4,5,5-tetramethyl-1,3,2-dioxaborolan-2-yl)phenyl)butanamido)-1H-pyrazole-1-carboxylate, 5 (10 g, 0.019 mol) and (E)-N-(5-bromopyridin-2-yl)-4-morpholinobut-2-enamide (7.7 g, 0.023 mol) in 1,4-Dioxane (100 mL) and water (40 mL) followed by Cs2CO3 (14.5 g, 0.045 mol) were added. The reaction mass was allowed to stir for 10 min with degassing and added Pd(PPh3)4 (1.1 g, heated the reaction mass for 4 h at 100° C. in a sealed tube. The reaction mass was cooled and diluted with brine solution. The aqueous layer was separated and re-extracted with ethyl acetate. The combined organic layer was evaporated to dryness and crude material was purified by silica column chromatography by eluting with 10%-15% methanol in DCM to get desired pure compound 44 (4.5g, 44%). LCMS: m/z=529.15 (M+H)+; HPLC: 95.17%, rt: 6.34 min.

Racemic (E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide was separated by using chiral preparative HPLC column (Method: Column: Chiral Pak IA (20 mm×250 mm, 5 micron), Elution: isocratic (50:50), A=ACN, B=MeOH, Flow: 20 mL/min) to afford the pure Isomer-1 and Isomer-2.

### Isomer-1 (Compound-44A)

1HNMR (DMSO-d6, 400 MHz): δ 12.02 (s, 1H), 10.78 (s, 1H), 10.44 (s, 1H), 8.61 (s, 1H), 8.28 (d, 1H), 8.07-8.05 (m, 1H), 7.68 (s, 1H), 7.57 (d, 1H), 7.41-7.37 (m, 2H), 6.81-6.78 (m, 1H), 6.49 (d, 1H), 6.13 (s, 1H), 3.61-3.58 (m, 4H), 3.36-3.34 (m, 1H), 3.12 (d, 2H), 2.41-2.32 (m, 1.82-1.76 (m, 1H), 0.97 (d, 3H), 0.88-0.85 (m, 2H), 0.67 (d, 3H), 0.62-0.59 (m, 2H); LCMS: m/z=529.15 (M+H)+; HPLC: 96.72%, rt: 6.39 min; Chiral HPLC: 97.68%, rt: 14.47.

### Isomer-2 (Compound-44B)

1HNMR (DMSO-d6, 400 MHz): δ 12.02 (s, 1H), 10.78 (s, 1H), 10.44 (s, 1H), 8.61 (s, 1H), 8.28 (d, 1H), 8.07-8.04 (m, 1H), 7.68 (s, 1H), 7.57 (d, 1H), 7.41-7.37 (m, 2H), 6.81-6.78 (m, 1H), 6.50 (d, 1H), 6.14 (s, 1H), 3.61-3.58 (m, 4H), 3.36-3.34 (m, 1H), 3.12 (d, 2H), 2.40-2.39 (m, 1.82-1.76 (m, 1H), 0.97 (d, 3H), 0.88-0.85 (m, 2H), 0.67 (d, 3H), 0.62-0.60 (m, 2H); LCMS: m/z=529.15 (M+H)+; HPLC: 96.24%, rt: 6.39 min; Chiral HPLC: 97.92%, rt: 8.80.

### Combination Studies of Compound-44A and Docetaxel

DU-145 cells were treated with serial dilutions of Compound-44A (CDK7 inhibitor), serial dilutions of docetaxel individually and in combination for 3 days. The effect of 3-day compound treatment on cell proliferation was measured using CTG reagent. Percentage inhibition of cell proliferation in individual (Compound-44A and docetaxel) and combination treatments (Compound-44A +docetaxel) were analyzed using CompuSyn software to assess synergy.

### RESULTS AND DISCUSSIONS

TABLE 2
Percent inhibition of proliferation
of DU 145 cells by docetaxel on Day-3

| Docetaxel | | | |
|---|---|---|---|
| nM | % inhibition in proliferation | | |
| 10000.00 | 118.7 | 118.3 | 117.0 |
| 3333.3333 | 123.6 | 124.1 | 121.7 |
| 1111.1111 | 124.7 | 125.2 | 121.8 |
| 370.3704 | 126.3 | 125.0 | 123.2 |
| 123.4568 | 125.9 | 126.0 | 122.3 |
| 41.1523 | 122.9 | 123.1 | 120.5 |
| 13.7174 | 115.2 | 117.2 | 114.3 |
| 4.5725 | 94.6 | 98.6 | 99.2 |
| 1.5242 | 49.8 | 57.5 | 59.4 |
| 0.5081 | 14.0 | 15.5 | 18.3 |
| 0.1694 | 8.3 | 2.2 | 4.1 |
| 0.0565 | 8.8 | 16.0 | 24.0 |
| 0.0188 | 0.8 | 0.9 | 10.0 |
| 0.0063 | −12.2 | −11.7 | 4.4 |
| 0.0021 | −2.5 | 1.2 | 3.4 |
| 0.0007 | −3.9 | −5.1 | 2.9 |
| 0.0002 | 1.6 | −0.5 | 3.2 |

TABLE 3
Percent inhibition of proliferation of
DU145 cells by Compound-44A on Day-3

| Compound-44A | | | |
|---|---|---|---|
| (nM) | % inhibition in proliferation | | |
| 10000.00 | 107.2 | 106.1 | 106.3 |
| 3333.3333 | 112.3 | 111.8 | 110.8 |
| 1111.1111 | 109.0 | 110.4 | 105.8 |
| 370.3704 | 74.1 | 77.9 | 80.7 |
| 123.4568 | 55.0 | 56.2 | 55.9 |
| 41.1523 | 31.2 | 30.9 | 25.2 |
| 13.7174 | 18.0 | 13.0 | 12.9 |
| 4.5725 | 5.1 | 5.9 | 3.4 |
| 1.5242 | 1.6 | −1.5 | 5.6 |
| 0.5081 | 8.2 | 0.7 | 3.1 |
| 0.1694 | −3.1 | 4.7 | −5.4 |
| 0.0565 | −1.2 | 8.1 | 1.7 |
| 0.01882 | −4.7 | 2.1 | −5.1 |
| 0.00627 | −1.3 | 3.7 | −0.3 |
| 0.00209 | 0.7 | −2.6 | 2.1 |

TABLE 4
Effect of combination of docetaxel and Compound-44A on proliferation of DU
145 cells: Checkerboard representing % inhibition in proliferation and combination
index. Combination index determined using Compusyn software, Combosyn inc., USA

| | | Compound-44A (nM) | | | | | | | |
|---|---|---|---|---|---|---|---|---|---|
| | | 0 | 0.5 | 1.5 | 4.5 | 13.7 | 41.1 | 123.4 | 370.3 |
| | Concentration | Numbers in unbold-% Inhibition in proliferation | | | | | | | |
| | (nM) | Numbers in Bold-Combination Index (CI) | | | | | | | |
| Docetaxel | 0 | 0.00 | 3.98 | 1.91 | 4.81 | 14.64 | 29.13 | 55.67 | 77.56 |
| (nM) | 0.0002 | 1.42 | 0.72 | 4.32 | 9.35 | 30.10 | 48.45 | 67.71 | 92.11 |
| | | | 12.93 | 1.64 | 0.61 | 0.15 | 0.13 | 0.18 | 0.29 |
| | 0.0007 | 0.00 | 2.79 | 6.37 | 9.34 | 29.23 | 49.12 | 66.38 | 91.50 |
| | | | 1.11 | 0.20 | 0.61 | 0.15 | 0.13 | 0.18 | 0.29 |
| | 0.0021 | 0.69 | 0.93 | 3.55 | 5.20 | 30.60 | 46.23 | 65.51 | 91.35 |
| | | | 16.49 | 1.72 | 0.61 | 0.15 | 0.13 | 0.18 | 0.29 |
| | 0.0063 | 0.00 | 2.83 | 6.31 | 6.88 | 30.99 | 45.97 | 66.48 | 89.15 |
| | | | 1.64 | 0.23 | 0.63 | 0.15 | 0.13 | 0.18 | 0.29 |
| | 0.0188 | 3.89 | 3.36 | 6.58 | 11.84 | 28.99 | 46.31 | 66.03 | 91.30 |
| | | | 2.85 | 0.27 | 0.67 | 0.15 | 0.13 | 0.18 | 0.29 |
| | 0.0565 | 16.29 | 5.00 | 9.79 | 12.95 | 30.09 | 48.76 | 65.93 | 95.40 |
| | | | 1.73 | 0.41 | 0.82 | 0.16 | 0.14 | 0.18 | 0.22 |
| | 0.1694 | 4.86 | 5.03 | 12.79 | 11.50 | 29.97 | 49.21 | 61.01 | 92.02 |
| | | | 0.71 | 0.84 | 1.25 | 0.18 | 0.14 | 0.26 | 0.29 |
| | 0.5081 | 15.93 | 12.74 | 20.59 | 20.87 | 37.51 | 53.05 | 71.81 | 101.78 |
| | | | 1.99 | 0.33 | 0.41 | 0.12 | 0.15 | 0.19 | 0.22 |
| | 1.52 | 55.58 | 61.83 | 64.69 | 59.44 | 75.00 | 81.58 | 97.36 | 108.51 |
| | | | 0.04 | 0.04 | 0.05 | 0.03 | 0.06 | 0.08 | 0.18 |
| | 4.57 | 97.48 | 100.56 | 105.55 | 104.33 | 111.08 | 106.77 | 114.57 | 117.95 |
| | | | 0.03 | 0.02 | 0.03 | 0.03 | 0.04 | 0.08 | 0.15 |
| | 13.72 | 115.54 | 122.58 | 123.74 | 123.94 | 123.20 | 118.13 | 124.74 | 124.84 |
| | | | 0.05 | 0.05 | 0.05 | 0.05 | 0.06 | 0.09 | 0.18 |
| | 41.15 | 122.18 | 128.28 | 129.17 | 129.19 | 127.22 | 122.11 | 127.88 | 126.74 |
| | | | 0.11 | 0.11 | 0.11 | 0.11 | 0.15 | 0.14 | 0.22 |
| | 123.46 | 124.73 | 128.22 | 128.92 | 129.30 | 127.57 | 122.01 | 128.24 | 126.98 |
| | | | 0.32 | 0.32 | 0.32 | 0.32 | 0.42 | 0.36 | 0.43 |
| | 370.37 | 124.85 | 128.72 | 129.42 | 129.62 | 128.19 | 121.68 | 128.35 | 127.04 |
| | | | 0.96 | 0.96 | 0.96 | 0.96 | 1.24 | 0.99 | 1.07 |
| | 1111.11 | 123.90 | 127.85 | 128.57 | 128.96 | 127.06 | 121.32 | 127.78 | 126.36 |
| | | | 2.87 | 2.87 | 2.87 | 2.87 | 3.69 | 2.90 | 2.98 |
| | 3333.33 | 123.13 | 126.64 | 127.17 | 127.22 | 125.49 | 120.08 | 126.47 | 125.77 |
| | | | 8.60 | 8.60 | 8.60 | 8.60 | 11.03 | 8.64 | 8.71 |
| | 10000.00 | 118.04 | 122.35 | 122.58 | 123.85 | 122.51 | 118.76 | 125.76 | 125.30 |
| | | | 33.04 | 33.04 | 33.05 | 33.05 | 33.06 | 25.83 | 25.91 |

TABLE 5
Synergistic effect of combination of docetaxel and Compound-44A on
proliferation of DU 145 cells

| | | | Compound-44A (nM) | | | | | |
|---|---|---|---|---|---|---|---|---|
| Concentration | | 0 | 1.5 | 4.5 | 13.7 | 41.1 | 123.4 | 370.3 |
| Docetaxel | 0 | 0 | 1.91 | 4.81 | 14.64 | 29.13 | 55.67 | 77.56 |
| (nM) | 0.17 | 4.86 | 12.79 | 11.50 | 29.97 | 49.21 | 61.01 | 92.02 |
| | 0.51 | 15.93 | 20.59 | 20.87 | 37.51 | 53.05 | 71.81 | 101.78 |
| | 1.52 | 55.58 | 64.69 | 59.44 | 75.00 | 81.58 | 97.36 | 108.51 |
| | 4.57 | 97.48 | 105.55 | 104.33 | 111.08 | 106.77 | 114.57 | 117.95 |
| | 13.72 | 115.54 | 123.74 | 123.94 | 123.20 | 118.13 | 124.74 | 124.84 |

### CONCLUSION

Compound-44A and Docetaxel showed dose dependent inhibition of proliferation in DU145 cells. Compound-44A showed synergy in combination with docetaxel in inhibiting the proliferation of prostate cancer cell line DU145.

### INCORPORATION BY REFERENCE

All publications and patents mentioned herein are hereby incorporated by reference in their entirety as if each individual publication or patent were specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating or preventing cancer in a subject, comprising administering to the subject a compound of formula (I): 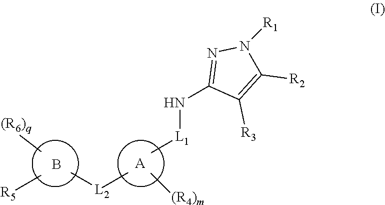
or a pharmaceutically acceptable salt thereof or a stereoisomer thereof, conjointly with an anti-microtubule agent;
wherein,
ring A is cycloalkyl, aryl, heteroaryl or heterocyclyl;
ring B is aryl, cycloalkyl, heterocyclyl or absent;
R₁ is hydrogen or alkyl;
R₂ is hydrogen, alkyl or cycloalkyl;
R₃ is hydrogen, alkyl or heteroaryl;
alternatively, R₂ together with R₁ or R₃ along with the ring atoms to which they are attached forms a 5-7 membered ring;
R₄ at each occurrence is halo, alkyl, hydroxy, alkoxy, amino, nitro, cyano or haloalkyl;
R₅ is 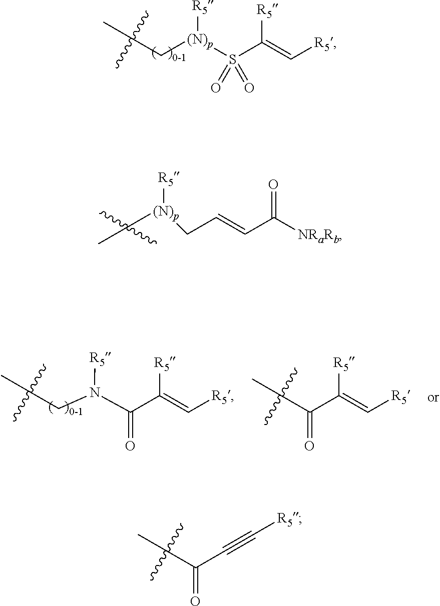
wherein R₄′ is hydrogen, halo, alkyl, alkoxy, alkoxyalkyl or —(CH₂)₁₋₃—NRₐR_{b}; R₅″ is H or alkyl;
Rₐ and R_{b} are each independently hydrogen, alkyl, alkoxy or alkoxyalkyl; alternatively, Rₐ and R_{b} together with the nitrogen atom to which they are attached form an optionally substituted ring containing 0-2 additional heteroatoms independently selected from N, O or S; wherein the optional substituent is one or more halo, alkyl, acyl, hydroxy, cyano, cyanoalkyl, haloalkyl, alkoxy, alkoxyalkyl, —COOH or —COO-alkyl;
R₆ at each occurrence is halo, alkyl, hydroxy, alkoxy, amino, nitro, cyano or haloalkyl;
L₁ is *—CR_{c}R_{d}—C(O)—, *—NRₑC(O)— or absent; wherein * is the point of attachment with ring A;
R_{c} and R_{d} independently are hydrogen, alkyl or haloalkyl; alternatively, R_{c} and R_{d} together with the carbon to which they are attached form a cycloalkyl ring;
Rₑ is hydrogen or alkyl;
L₂ is —C(O)NH—, —C(O)O— or absent;
m is 0, 1 or 2;
p is 0 or 1; and
q is 0 to 3.

2. The method of claim 1, wherein the compound is represented by formula (IA): 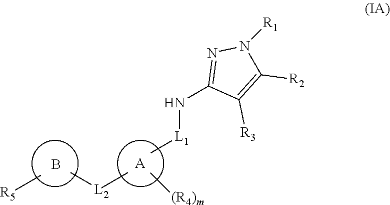
or a pharmaceutically acceptable salt thereof or a stereoisomer thereof;
wherein,
ring A is cycloalkyl, aryl, heteroaryl or heterocyclyl;
ring B is aryl, cycloalkyl, heterocyclyl or absent;
R₁ is hydrogen or alkyl;
R₂ is hydrogen, alkyl or cycloalkyl;
R₃ is hydrogen, alkyl or heteroaryl;
alternatively, R₂ together with R₁ or R₃ along with the ring atoms to which they are attached forms a 5-7 membered ring;
R4 at each occurrence is halo, alkyl, hydroxy or alkoxy;
R₅ is 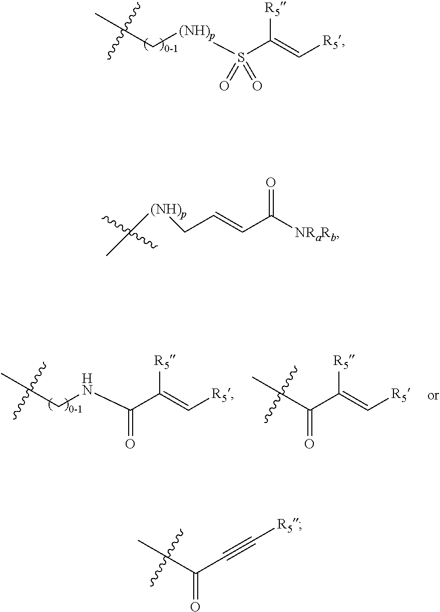
wherein R₅′ is hydrogen, halo, alkyl, alkoxy, alkoxyalkyl or —(CH₂)₁₋₃—NRₐR_{b}; R₅″ is H or alkyl;
Rₐ and R_{b} are each independently hydrogen or alkyl; alternatively, Rₐ and R_{b} together with the nitrogen atom to which they are attached form an optionally substituted ring containing 0-2 additional heteroatoms independently selected from N, O or S; wherein the optional substituent is one or more halo, alkyl, hydroxy, haloalkyl or alkoxy;
L₁ is *—CR_{c}R_{d}—C(O)—, *—NR,C(O)— or absent; wherein * is the point of attachment with ring A;
R_{c} and R_{d} independently are hydrogen, alkyl or haloalkyl; alternatively, R_{c} and R_{d} together with the carbon to which they are attached form a cycloalkyl ring;
Rₑ is hydrogen or alkyl;
L₂ is —C(O)NH—, —C(O)O— or absent;
m is 0, 1 or 2; and
p is 0 or 1.

3. The method of claim 1, wherein the compound is represented by formula (IB): 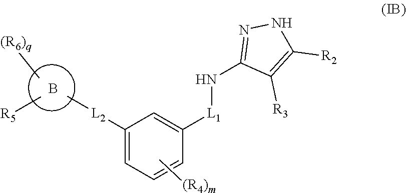
or a pharmaceutically acceptable salt thereof or a stereoisomer thereof.

4. The method of claim 1, wherein the compound is represented by formula (IC): 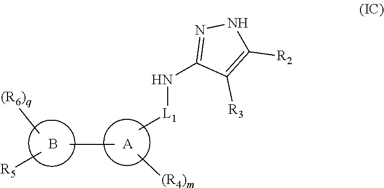
or a pharmaceutically acceptable salt thereof or a stereoisomer thereof.

5. The method of claim 1, wherein the compound is represented by formula (ID): 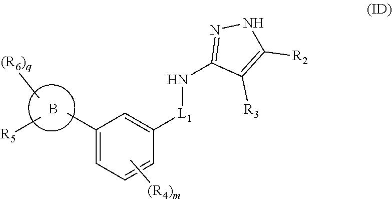
or a pharmaceutically acceptable salt thereof or a stereoisomer thereof.

6. The method of claim 1, wherein the compound is represented by formula (IE): 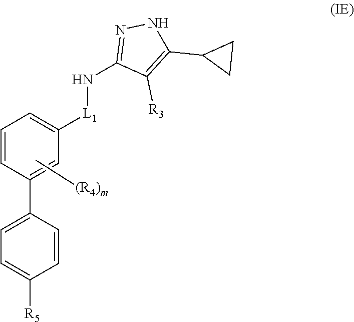
or a pharmaceutically acceptable salt thereof or a stereoisomer thereof;
wherein, L₁ is *—CR_{c}R_{d}—C(O)— or *—NRₑC(O)—; wherein * is the point of attachment with the phenyl ring.

7. The method of claim 1, wherein the compound is represented by formula (IF): 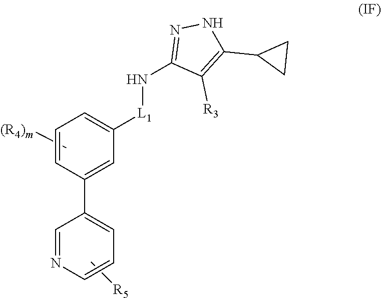
or a pharmaceutically acceptable salt thereof or a stereoisomer thereof;
wherein, L₁ is *—CR_{c}R_{d}—C(O)— or *—NRₑC(O)—; wherein * is the point of attachment with the phenyl ring.

8. The method of claim 1, wherein the compound is represented by formula (IG): 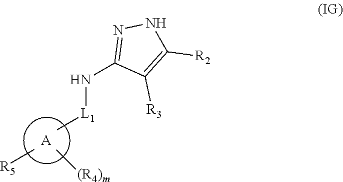
or a pharmaceutically acceptable salt thereof or a stereoisomer thereof.

9. The method of claim 1, wherein ring A is aryl or heteroaryl.

10. (canceled)

11. The method of claim 1, wherein ring B is monocyclic or bicyclic, cycloalkyl, aryl, heterocycloalkyl or heteroaryl.

12. The method of claim 1, wherein R₂ is cycloalkyl.

13. The method of claim 1, wherein R₅ is

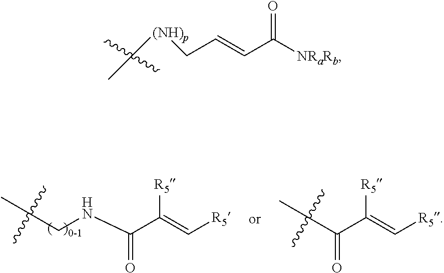

14.-15. (canceled)

16. The method of claim 1, wherein L₁ is *—CR_{c}R_{d}—C(O)—; wherein * is the point of attachment with ring A.

17. (canceled)

18. The method of claim 1, wherein the compound of formula (I) is selected from:

| Compound | |
|---|---|
| No. | IUPAC name |
| 1 | (E)-N-(3′-(1-((5-cyclopropyl-1H-pyrazol-3-yl)amino)-1-oxopropan-2-yl)-[1,1′- |
| | biphenyl]-4-yl)-4-(dimethylamino)but-2-enamide; |
| 2 | (E)-N-(3′-(1-((5-cyclopropyl-1H-pyrazol-3-yl)amino)-1-oxopropan-2-yl)-[1,1′- |
| | biphenyl]-4-yl)-4-(dimethylamino)but-2-enamide (Isomer-1 of compound-1); |
| 3 | (E)-N-(3′-(1-((5-cyclopropyl-1H-pyrazol-3-yl)amino)-1-oxopropan-2-yl)-[1,1′- |
| | biphenyl]-4-yl)-4-(dimethylamino)but-2-enamide (Isomer-2 of compound-1); |
| 4 | (E)-N-(5′-(1-((5-cyclopropyl-1H-pyrazol-3-yl)amino)-1-oxopropan-2-yl)-2′-fluoro- |
| | [1,1′-biphenyl]-4-yl)-4-(dimethylamino)but-2-enamide; |
| 5 | (E)-N-(3′-(1-((5-cyclopropyl-1H-pyrazol-3-yl)amino)-1-oxopropan-2-yl)-4′-fluoro- |
| | [1,1′-biphenyl]-4-yl)-4-(dimethylamino)but-2-enamide; |
| 7 | (E)-N-(3′-(2-((5-cyclopropyl-1H-pyrazol-3-yl)amino)-2-oxoethyl)-[1,1′-biphenyl]-4- |
| | yl)-4-(dimethylamino)but-2-enamide; |
| 8 | (E)-4-(dimethylamino)-N-(3′-(1-((5-ethyl-1H-pyrazol-3-yl)amino)-1-oxopropan-2- |
| | yl)-[1,1′-biphenyl]-4-yl)but-2-enamide; |
| 9 | (E)-N-(3′-(1-((5-(tert-butyl)-1H-pyrazol-3-yl)amino)-1-oxopropan-2-yl)-[1,1′- |
| | biphenyl]-4-yl)-4-(dimethylamino)but-2-enamide; |
| 10 | (E)-N-(5-(3-(1-((5-cyclopropyl-1H-pyrazol-3-yl)amino)-1-oxopropan-2- |
| | yl)phenyl)pyridin-2-yl)-4-morpholinobut-2-enamide; |
| 11 | (E)-N-(5-(3-(1-((5-cyclopropyl-1H-pyrazol-3-yl)amino)-1-oxopropan-2- |
| | yl)phenyl)pyridin-2-yl)-4-morpholinobut-2-enamide (Isomer-1 of compound-10); |
| 12 | (E)-N-(5-(3-(1-((5-cyclopropyl-1H-pyrazol-3-yl)amino)-1-oxopropan-2- |
| | yl)phenyl)pyridin-2-yl)-4-morpholinobut-2-enamide (Isomer-2 of compound-10); |
| 13 | (E)-N-(3′-(1-((5-cyclopropyl-1H-pyrazol-3-yl)amino)-1-oxobutan-2-yl)-[1,1′- |
| | biphenyl]-4-yl)-4-(dimethylamino)but-2-enamide; |
| 14 | (E)-N-(3′-(1-((5-cyclopropyl-1H-pyrazol-3-yl)amino)-1-oxopropan-2-yl)-3-fluoro- |
| | [1,1′-biphenyl]-4-yl)-4-(dimethylamino)but-2-enamide; |
| 15 | (E)-N-(3′-(1-((5-cyclobutyl-1H-pyrazol-3-yl)amino)-1-oxopropan-2-yl)-[1,1′- |
| | biphenyl]-4-yl)-4-(dimethylamino)but-2-enamide; |
| 16 | (E)-N-(6-(3-(1-((5-cyclopropyl-1H-pyrazol-3-yl)amino)-1-oxopropan-2- |
| | yl)phenyl)pyridin-3-yl)-4-(dimethylamino)but-2-enamide; |
| 17 | (E)-N-(3′-(1-((5-cyclopropyl-1H-pyrazol-3-yl)amino)-1-oxopropan-2-yl)-2-fluoro- |
| | [1,1′-biphenyl]-4-yl)-4-(dimethylamino)but-2-enamide; |
| 18 | N-(3′-(1-((5-cyclopropyl-1H-pyrazol-3-yl)amino)-1-oxopropan-2-yl)-[1,1′- |
| | biphenyl]-4-yl)acrylamide; |
| 19 | N-(3′-(1-((5-cyclopropyl-1H-pyrazol-3-yl)amino)-1-oxopropan-2-yl)-3-fluoro-[1,1′- |
| | biphenyl]-4-yl)acrylamide; |
| 20 | N-(5-(3-(1-((5-cyclopropyl-1H-pyrazol-3-yl)amino)-1-oxopropan-2- |
| | yl)phenyl)pyridin-2-yl)acrylamide; |
| 21 | N-(5-(3-(1-((5-cyclopropyl-1H-pyrazol-3-yl)amino)-1-oxopropan-2- |
| | yl)phenyl)pyridin-2-yl)acrylamide (Isomer-1 of compound-20); |
| 22 | N-(5-(3-(1-((5-cyclopropyl-1H-pyrazol-3-yl)amino)-1-oxopropan-2- |
| | yl)phenyl)pyridin-2-yl)acrylamide (Isomer-2 of compound-20); |
| 23 | N-(5′-(1-((5-cyclopropyl-1H-pyrazol-3-yl)amino)-1-oxopropan-2-yl)-2′,3-difluoro- |
| | [1,1′-biphenyl]-4-yl)acrylamide; |
| 24 | N-(6-(3-(1-((5-cyclopropyl-1H-pyrazol-3-yl)amino)-1-oxopropan-2- |
| | yl)phenyl)pyridin-3-yl)acrylamide; |
| 25 | 2-(4′-acrylamido-3′-fluoro-[1,1′-biphenyl]-3-yl)-N-(5-cyclopropyl-1H-pyrazol-3-yl)- |
| | 3-methylbutanamide; |
| 26 | 2-(3-(5-acrylamidopyridin-2-yl)phenyl)-N-(5-cyclopropyl-1H-pyrazol-3- |
| | yl)butanamide; |
| 27 | 2-(4′-acrylamido-3′-fluoro-[1,1′-biphenyl]-3-yl)-N-(5-cyclopropyl-1H-pyrazol-3- |
| | yl)butanamide; |
| 28 | (E)-N-(3′-(1-((5-cyclopentyl-1H-pyrazol-3-yl)amino)-1-oxopropan-2-yl)-3-fluoro- |
| | [1,1′-biphenyl]-4-yl)-4-(dimethylamino)but-2-enamide; |
| 29 | (E)-N-(5-cyclopropyl-1H-pyrazol-3-yl)-2-(3-(1-(4-(dimethylamino)but-2- |
| | enoyl)indolin-5-yl)phenyl)propanamide; |
| 30 | N-(3′-(1-((5-cyclopropyl-4-methyl-1H-pyrazol-3-yl)amino)-1-oxopropan-2-yl)-[1,1′- |
| | biphenyl]-4-yl)acrylamide; |
| 31 | (E)-N-(3′-(1-((5-cyclopropyl-1H-pyrazol-3-yl)amino)-1-oxobutan-2-yl)-3-fluoro- |
| | [1,1′-biphenyl]-4-yl)-4-(dimethylamino)but-2-enamide; |
| 32 | (E)-N-(3′-(1-((5-cyclopropyl-1H-pyrazol-3-yl)amino)-1-oxopropan-2-yl)-3-fluoro- |
| | [1,1′-biphenyl]-4-yl)-4-(diethylamino)but-2-enamide; |
| 33 | (E)-N-(3′-(1-((5-cyclopropyl-1H-pyrazol-3-yl)amino)-1-oxopropan-2-yl)-3-fluoro- |
| | [1,1′-biphenyl]-4-yl)-4-(pyrrolidin-1-yl)but-2-enamide; |
| 34 | (E)-N-(3′-(1-((5-cyclopropyl-1H-pyrazol-3-yl)amino)-1-oxopropan-2-yl)-3-fluoro- |
| | [1,1′-biphenyl]-4-yl)-4-morpholinobut-2-enamide; |
| 35 | (E)-N-(6-(3-(1-((5-cyclopropyl-1H-pyrazol-3-yl)amino)-1-oxopropan-2- |
| | yl)phenyl)pyridin-3-yl)-4-morpholinobut-2-enamide; |
| 36 | (E)-N-(6-(3-(1-((5-cyclopropyl-1H-pyrazol-3-yl)amino)-3-methyl-1-oxobutan-2- |
| | yl)phenyl)pyridin-3-yl)-4-(dimethylamino)but-2-enamide; |
| 37 | (E)-4-((6-(3-(1-((5-cyclopropyl-1H-pyrazol-3-yl)amino)-1-oxopropan-2- |
| | yl)phenyl)pyridin-3-yl)amino)-N,N-dimethylbut-2-enamide; |
| 38 | (E)-N-(6-(3-(1-((5-cyclopropyl-1H-pyrazol-3-yl)amino)-1-oxopropan-2-yl)phenyl)- |
| | 2-fluoropyridin-3-yl)-4-(dimethylamino)but-2-enamide; |
| 39 | (E)-N-(6-(3-(1-((5-cyclopropyl-1H-pyrazol-3-yl)amino)-1-oxopropan-2- |
| | yl)phenyl)pyridin-3-yl)-4-(piperidin-1-yl)but-2-enamide; |
| 40 | (E)-N-(5-(3-(1-((5-cyclopropyl-1H-pyrazol-3-yl)amino)-1-oxopropan-2- |
| | yl)phenyl)pyridin-2-yl)-4-(piperidin-1-yl)but-2-enamide; |
| 41 | (E)-N-(5-(3-(1-((5-cyclopropyl-1H-pyrazol-3-yl)amino)-1-oxopropan-2- |
| | yl)phenyl)pyridin-2-yl)-4-methoxybut-2-enamide; |
| 42 | (E)-N-(5-(3-(1-((5-cyclopropyl-1H-pyrazol-3-yl)amino)-1-oxopropan-2- |
| | yl)phenyl)pyridin-2-yl)-4-(dimethylamino)but-2-enamide; |
| 43 | (E)-N-(3′-(1-((5-cyclopropyl-1H-pyrazol-3-yl)amino)-3-methyl-1-oxobutan-2-yl)-3- |
| | fluoro-[1,1′-biphenyl]-4-yl)-4-(dimethylamino)but-2-enamide; |
| 44 | (E)-N-(5-(3-(1-((5-cyclopropyl-1H-pyrazol-3-yl)amino)-3-methyl-1-oxobutan-2- |
| | yl)phenyl)pyridin-2-yl)-4-morpholinobut-2-enamide; |
| 44A | (S)-(E)-N-(5-(3-(1-((5-cyclopropyl-1H-pyrazol-3-yl)amino)-3-methyl-1-oxobutan-2- |
| | yl)phenyl)pyridin-2-yl)-4-morpholinobut-2-enamide; |
| 44B | (R)-(E)-N-(5-(3-(1-((5-cyclopropyl-1H-pyrazol-3-yl)amino)-3-methyl-1-oxobutan- |
| | 2-yl)phenyl)pyridin-2-yl)-4-morpholinobut-2-enamide; |
| 45 | (E)-N-(5-(3-(1-((5-cyclopropyl-1H-pyrazol-3-yl)amino)-1-oxopropan-2- |
| | yl)phenyl)pyridin-2-yl)-4-(3-fluoropyrrolidin-1-yl)but-2-enamide; |
| 46 | (E)-N-(5-(3-(1-((5-cyclopropyl-1H-pyrazol-3-yl)amino)-1-oxopropan-2- |
| | yl)phenyl)pyridin-2-yl)-4-((S)-3-fluoropyrrolidin-1-yl)but-2-enamide; |
| 47 | (E)-N-(5-(3-(1-((5-cyclopropyl-1H-pyrazol-3-yl)amino)-1-oxopropan-2- |
| | yl)phenyl)pyridin-2-yl)-4-((S)-3-fluoropyrrolidin-1-yl)but-2-enamide (Isomer-1 of |
| | compound-46); |
| 48 | E)-N-(5-(3-(1-((5-cyclopropyl-1H-pyrazol-3-yl)amino)-1-oxopropan-2- |
| | yl)phenyl)pyridin-2-yl)-4-((S)-3-fluoropyrrolidin-1-yl)but-2-enamide (Isomer-2 of |
| | compound-46); |
| 49 | (E)-N-(5-(3-(1-((5-cyclopropyl-1H-pyrazol-3-yl)amino)-1-oxopropan-2- |
| | yl)phenyl)pyridin-2-yl)-4-((R)-3-fluoropyrrolidin-1-yl)but-2-enamide |
| 50 | (E)-N-(5-(3-(1-((5-cyclopropyl-1H-pyrazol-3-yl)amino)-1-oxopropan-2- |
| | yl)phenyl)pyridin-2-yl)-4-((R)-3-fluoropyrrolidin-1-yl)but-2-enamide (Isomer-1 of |
| | compound-49); |
| 51 | (E)-N-(5-(3-(1-((5-cyclopropyl-1H-pyrazol-3-yl)amino)-1-oxopropan-2- |
| | yl)phenyl)pyridin-2-yl)-4-((R)-3-fluoropyrrolidin-1-yl)but-2-enamide (Isomer-2 of |
| | compound-49); |
| 52 | (E)-N-(5-(3-(1-((5-cyclopropyl-1H-pyrazol-3-yl)amino)-1-oxopropan-2- |
| | yl)phenyl)pyridin-2-yl)-4-(pyrrolidin-1-yl)but-2-enamide; |
| 53 | (E)-N-(5-(3-(1-((5-cyclopropyl-1H-pyrazol-3-yl)amino)-1-oxopropan-2-yl)- |
| | phenyl)pyridin-2-yl)-4-(pyrrolidin-1-yl)but-2-enamide (Isomer-1 of compound-52); |
| 54 | (E)-N-(5-(3-(1-((5-cyclopropyl-1H-pyrazol-3-yl)amino)-1-oxopropan-2-yl)- |
| | phenyl)pyridin-2-yl)-4-(pyrrolidin-1-yl)but-2-enamide (Isomer-2 of compound-52); |
| 55 | (E)-N-(5-(3-(1-((5-cyclopropyl-1H-pyrazol-3-yl)amino)-1-oxopropan-2- |
| | yl)phenyl)pyridin-2-yl)-4-(diethylamino)but-2-enamide; |
| 56 | (E)-N-(5-(3-(1-((5-cyclopropyl-1H-pyrazol-3-yl)amino)-3-methyl-1-oxobutan-2- |
| | yl)phenyl)pyridin-2-yl)-4-(dimethylamino)but-2-enamide; |
| 57 | (E)-N-(5-(3-(1-((5-cyclopropyl-1H-pyrazol-3-yl)amino)-1-oxopropan-2- |
| | yl)phenyl)pyridin-2-yl)-4-(3-fluoropiperidin-1-yl)but-2-enamide; |
| 58 | (E)-N-(6-(3-(1-((5-cyclopropyl-1H-pyrazol-3-yl)amino)-1-oxopropan-2-yl)phenyl)- |
| | 2-fluoropyridin-3-yl)-4-morpholinobut-2-enamide; |
| 59 | (E)-N-(5-(3-(1-((5-cyclopropyl-1H-pyrazol-3-yl)amino)-1-oxopropan-2-yl)phenyl)- |
| | 6-fluoropyridin-2-yl)-4-morpholinobut-2-enamide; |
| 60 | (E)-N-(5-(3-(1-((5-cyclopropyl-1H-pyrazol-3-yl)amino)-1-oxopropan-2- |
| | yl)phenyl)pyridin-3-yl)-4-morpholinobut-2-enamide; |
| 61 | (E)-N-(3′-(1-((5-cyclopropyl-1H-pyrazol-3-yl)amino)-1-oxopropan-2-yl)-[1,1′- |
| | biphenyl]-3-yl)-4-morpholinobut-2-enamide; |
| 62 | (E)-N-(5-(3-(1-((5-cyclopropyl-1H-pyrazol-3-yl)amino)-1-oxopropan-2- |
| | yl)phenyl)pyridin-2-yl)-4-(methoxy(methyl)amino)but-2-enamide; |
| 63 | (E)-N-(5-(3-(1-((5-cyclopropyl-1H-pyrazol-3-yl)amino)-1-oxopropan-2-yl)phenyl)- |
| | 3-fluoropyridin-2-yl)-4-morpholinobut-2-enamide; |
| 64 | (E)-N-(6-(3-(1-((5-cyclopropyl-1H-pyrazol-3-yl)amino)-1-oxopropan-2-yl)phenyl)- |
| | 2-fluoropyridin-3-yl)-4-(3-fluoropyrrolidin-1-yl)but-2-enamide; |
| 65 | N-(3′-(1-((5-cyclopropyl-1H-pyrazol-3-yl)amino)-1-oxopropan-2-yl)-3-methoxy- |
| | [1,1′-biphenyl]-4-yl)acrylamide; |
| 66 | N-(3′-(1-((5-cyclopropyl-1H-pyrazol-3-yl)amino)-1-oxopropan-2-yl)-3-methyl-[1,1′- |
| | biphenyl]-4-yl)acrylamide; |
| 67 | N-(3′-(1-((5-cyclopropyl-1H-pyrazol-3-yl)amino)-1-oxopropan-2-yl)-3,5-dimethyl- |
| | [1,1′-biphenyl]-4-yl)acrylamide; |
| 68 | (E)-N-(5-(3-(1-((5-cyclopropyl-1H-pyrazol-3-yl)amino)-1-oxopropan-2- |
| | yl)phenyl)pyridin-2-yl)-4-((2-methoxyethyl)(methyl)amino)but-2-enamide; |
| 69 | (E)-N-(5-(3-(1-((5-cyclopropyl-1H-pyrazol-3-yl)amino)-1-oxopropan-2- |
| | yl)phenyl)pyridin-2-yl)-4-(1H-imidazol-1-yl)but-2-enamide; |
| 70 | (E)-N-(5-(3-(1-((5-cyclopropyl-1H-pyrazol-3-yl)amino)-1-oxopropan-2- |
| | yl)phenyl)pyridin-2-yl)-4-((S)-2-(methoxymethyl)pyrrolidin-1-yl)but-2-enamide; |
| 71 | (E)-N-(5-(3-(1-((5-cyclopropyl-1H-pyrazol-3-yl)amino)-1-oxopropan-2- |
| | yl)phenyl)pyridin-2-yl)-4-((S)-2-(methoxymethyl)pyrrolidin-1-yl)but-2-enamide |
| | (Isomer-1 of compound-70); |
| 72 | (E)-N-(5-(3-(1-((5-cyclopropyl-1H-pyrazol-3-yl)amino)-1-oxopropan-2- |
| | yl)phenyl)pyridin-2-yl)-4-((S)-2-(methoxymethyl)pyrrolidin-1-yl)but-2-enamide |
| | (Isomer-2 of compound-70); |
| 73 | N-(3′-(1-((5-cyclopropyl-1H-pyrazol-3-yl)amino)-1-oxopropan-2-yl)-2-methoxy- |
| | [1,1′-biphenyl]-4-yl)acrylamide; |
| 74 | N-((5-(3-(1-((5-cyclopropyl-1H-pyrazol-3-yl)amino)-1-oxopropan-2- |
| | yl)phenyl)pyridin-2-yl)methyl)acrylamide; |
| 75 | N-(3′-(1-((5-cyclopropyl-1H-pyrazol-3-yl)amino)-1-oxopropan-2-yl)-2-methyl-[1,1′- |
| | biphenyl]-4-yl)acrylamide; |
| 76 | 2-(3-(1-acryloylindolin-5-yl)phenyl)-N-(5-cyclopropyl-1H-pyrazol-3- |
| | yl)propanamide; |
| 77 | (E)-N-(5-(3-(1-((5-cyclopropyl-1H-pyrazol-3-yl)amino)-1-oxopropan-2- |
| | yl)phenyl)pyridin-2-yl)-4-((2S,4S)-4-fluoro-2-(methoxymethyl)pyrrolidin-1-yl)but- |
| | 2-enamide; |
| 78 | (E)-4-(3-cyanopyrrolidin-1-yl)-N-(5-(3-(1-((5-cyclopropyl-1H-pyrazol-3-yl)amino)- |
| | 1-oxopropan-2-yl)phenyl)pyridin-2-yl)but-2-enamide; |
| 79 | N-(6-(3-(1-((5-cyclopropyl-1H-pyrazol-3-yl)amino)-1-oxopropan-2- |
| | yl)phenyl)pyrazin-2-yl)acrylamide; |
| 80 | (E)-N-(3′-(1-((5-cyclopropyl-1H-pyrazol-3-yl)amino)-1-oxopropan-2-yl)-3-fluoro- |
| | [1,1′-biphenyl]-4-yl)-4-((S)-3-fluoropyrrolidin-1-yl)but-2-enamide; |
| 81 | methyl ((E)-4-((5-(3-(1-((5-cyclopropyl-1H-pyrazol-3-yl)amino)-1-oxopropan-2- |
| | yl)phenyl)pyridin-2-yl)amino)-4-oxobut-2-en-1-yl)-L-prolinate; |
| 82 | (E)-4-((S)-2-(cyanomethyl)pyrrolidin-1-yl)-N-(5-(3-(1-((5-cyclopropyl-1H-pyrazol- |
| | 3-yl)amino)-1-oxopropan-2-yl)phenyl)pyridin-2-yl)but-2-enamide; |
| 83 | 4-acrylamido-N-(3-((5-ethyl-1H-pyrazol-3-yl)amino)phenyl)benzamide; |
| 84 | (E)-N-(5-cyclopropyl-1H-pyrazol-3-yl)-2-(3-(1-(4-(dimethylamino)but-2-enoyl)- |
| | 1,2,3,6-tetrahydropyridin-4-yl)phenyl)propanamide; |
| 85 | (E)-N-(5-cyclopropyl-1H-pyrazol-3-yl)-2-(3-(1-(4-(dimethylamino)but-2- |
| | enoyl)piperidin-4-yl)phenyl)propanamide; |
| 86 | N-(3′-(2-((5-methyl-1H-pyrazol-3-yl)amino)-2-oxoethyl)-[1,1′-biphenyl]-4- |
| | yl)acrylamide; |
| 87 | N-(3′-(1-((5-ethyl-1H-pyrazol-3-yl)amino)-1-oxopropan-2-yl)-[1,1′-biphenyl]-4- |
| | yl)acrylamide; |
| 88 | N-(3′-(1-((5-(tert-butyl)-1H-pyrazol-3-yl)amino)-1-oxopropan-2-yl)-[1,1′-biphenyl]- |
| | 4-yl)acrylamide; |
| 89 | (E)-N-(3-((1H-indazol-3-yl)amino)phenyl)-4-(4-(dimethylamino)but-2- |
| | enamido)benzamide; |
| 90 | N-(3′-(1-((1H-indazol-3-yl)amino)-1-oxopropan-2-yl)-[1,1′-biphenyl]-4- |
| | yl)acrylamide; |
| 91 | (E)-N-(3′-(1-((1H-indazol-3-yl)amino)-1-oxopropan-2-yl)-[1,1′-biphenyl]-4-yl)-4- |
| | (dimethylamino)but-2-enamide; |
| 92 | (E)-N-(6-(3-(1-((5-cyclopropyl-1H-pyrazol-3-yl)amino)-1-oxopropan-2- |
| | yl)phenyl)pyrazin-2-yl)-4-(pyrrolidin-1-yl)but-2-enamide; |
| 93 | (S,E)-N-(5-(3-(1-((5-cyclopropyl-1H-pyrazol-3-yl)amino)-1-oxopropan-2- |
| | yl)phenyl)pyridin-2-yl)-4-morpholinobut-2-enamide; |
| 94 | (E)-N-(5-cyclopropyl-1H-pyrazol-3-yl)-1-(3-(6-(4-(pyrrolidin-1-yl)but-2- |
| | enamido)pyridin-3-yl)phenyl)cyclopropane-1-carboxamide; |
| 95 | (E)-N-(5-(3-(1-((5-cyclopropyl-1H-pyrazol-3-yl)amino)-2-methyl-1-oxopropan-2- |
| | yl)phenyl)pyridin-2-yl)-4-(pyrrolidin-1-yl)but-2-enamide; |
| 96 | (E)-N-(5-(3-(1-((5-cyclopropyl-1H-pyrazol-3-yl)amino)-3-methyl-1-oxobutan-2- |
| | yl)phenyl)pyridin-2-yl)-4-(pyrrolidin-1-yl)but-2-enamide; |
| 97 | (E)-N-(5-(3-(1-((5-cyclopropyl-1H-pyrazol-3-yl)amino)-3-methyl-1-oxobutan-2- |
| | yl)phenyl)pyridin-2-yl)-4-(3-fluoropyrrolidin-1-yl)but-2-enamide; |
| 98 | (E)-N-(5-(3-(1-((5-cyclopropyl-1H-pyrazol-3-yl)amino)-3-methyl-1-oxobutan-2- |
| | yl)phenyl)pyridin-2-yl)-4-(3-hydroxypyrrolidin-1-yl)but-2-enamide; |
| 99 | (E)-N-(5-(3-(1-((5-cyclopropyl-1H-pyrazol-3-yl)amino)-3-methyl-1-oxobutan-2- |
| | yl)phenyl)pyridin-2-yl)-4-(piperidin-1-yl)but-2-enamide; |
| 100 | (E)-N-(5-(3-(1-((5-cyclopropyl-1H-pyrazol-3-yl)amino)-3-methyl-1-oxobutan-2- |
| | yl)phenyl)pyridin-2-yl)-4-(3-fluoropiperidin-1-yl)but-2-enamide; |
| 101 | (E)-N-(5-(3-(1-((5-cyclopropyl-1H-pyrazol-3-yl)amino)-3-methyl-1-oxobutan-2- |
| | yl)phenyl)pyridin-2-yl)-4-(3-hydroxypiperidin-1-yl)but-2-enamide; |
| 102 | (E)-N-(5-(3-(1-((5-cyclopropyl-1H-pyrazol-3-yl)amino)-3-methyl-1-oxobutan-2- |
| | yl)phenyl)pyridin-2-yl)-4-(4-fluoropiperidin-1-yl)but-2-enamide; |
| 103 | (E)-N-(5-(3-(1-((5-cyclopropyl-1H-pyrazol-3-yl)amino)-3-methyl-1-oxobutan-2- |
| | yl)phenyl)pyridin-2-yl)-4-(4-hydroxy-4-methylpiperidin-1-yl)but-2-enamide; |
| 104 | (E)-N-(5-(3-(1-((5-cyclopropyl-1H-pyrazol-3-yl)amino)-3-methyl-1-oxobutan-2- |
| | yl)phenyl)pyridin-2-yl)-4-(4,4-difluoropiperidin-1-yl)but-2-enamide; |
| 105 | (E)-N-(5-(3-(1-((5-cyclopropyl-1H-pyrazol-3-yl)amino)-3-methyl-1-oxobutan-2- |
| | yl)phenyl)pyridin-2-yl)-4-(diethylamino)but-2-enamide; |
| 106 | (E)-N-(1-(3-(1-((5-cyclopropyl-1H-pyrazol-3-yl)amino)-1-oxopropan-2- |
| | yl)phenyl)piperidin-4-yl)-4-(dimethylamino)but-2-enamide; |
| 107 | (E)-N-(3-(3-(1-((5-cyclopropyl-1H-pyrazol-3-yl)amino)-1-oxopropan-2-yl)phenyl)- |
| | 1-methyl-1H-pyrazol-5-yl)-4-(dimethylamino)but-2-enamide; |
| 108 | (E)-N-(5-cyclopropyl-1H-pyrazol-3-yl)-2-(3-(4-(4-(dimethylamino)but-2- |
| | enoyl)piperazin-1-yl)phenyl)propanamide; |
| 109 | 3-(1-((5-cyclopropyl-1H-pyrazol-3-yl)amino)-1-oxopropan-2-yl)phenyl (E)-4-(4- |
| | (dimethylamino)but-2-enoyl)piperazine-1-carboxylate; |
| 110 | (E)-4-((S)-2-cyanopyrrolidin-1-yl)-N-(5-(3-((S)-1-((5-cyclopropyl-1H-pyrazol-3- |
| | yl)amino)-1-oxopropan-2-yl)phenyl)pyridin-2-yl)but-2-enamide; |
| 111 | (E)-4-((2S,4S)-2-(cyanomethyl)-4-fluoropyrrolidin-1-yl)-N-(5-(3-((S)-1-((5- |
| | cyclopropyl-1H-pyrazol-3-yl)amino)-1-oxopropan-2-yl)phenyl)pyridin-2-yl)but-2- |
| | enamide; |
| 112 | (E)-N-(3′-((S)-1-((5-cyclopropyl-1H-pyrazol-3-yl)amino)-1-oxopropan-2-yl)-3- |
| | fluoro-[1,1′-biphenyl]-4-yl)-4-((S)-2-(methoxymethyl)pyrrolidin-1-yl)but-2- |
| | enamide; |
| 113 | (E)-N-(3-(1-((5-cyclopropyl-1H-pyrazol-3-yl)amino)-1-oxopropan-2-yl)phenyl)-4- |
| | (4-(dimethylamino)but-2-enoyl)morpholine-2-carboxamide; |
| 114 | (E)-N-(3-(1-((5-cyclopropyl-1H-pyrazol-3-yl)amino)-1-oxopropan-2-yl)phenyl)-4- |
| | (4-(dimethylamino)but-2-enamido)tetrahydro-2H-pyran-3-carboxamide; |
| 115 | (E)-N-(3-(1-((5-cyclopropyl-1H-pyrazol-3-yl)amino)-1-oxopropan-2-yl)phenyl)-1- |
| | (4-(dimethylamino)but-2-enoyl)pyrrolidine-2-carboxamide; |
| 116 | (E)-N-(3-(1-((5-cyclopropyl-1H-pyrazol-3-yl)amino)-1-oxopropan-2-yl)phenyl)-1- |
| | (4-(dimethylamino)but-2-enoyl)piperidine-2-carboxamide; |
| 117 | (E)-N-(3-(1-((5-cyclopropyl-1H-pyrazol-3-yl)amino)-1-oxopropan-2-yl)phenyl)-1- |
| | (4-(dimethylamino)but-2-enoyl)piperidine-3-carboxamide; |
| 118 | (E)-N-(3-(1-((5-cyclopropyl-1H-pyrazol-3-yl)amino)-1-oxopropan-2-yl)phenyl)-4- |
| | (dimethylamino)but-2-enamide; |
| 119 | (E)-N-(4-(1-((5-cyclopropyl-1H-pyrazol-3-yl)amino)-1-oxopropan-2-yl)phenyl)-4- |
| | (dimethylamino)but-2-enamide; |
| 120 | (E)-N-(2-(1-((5-cyclopropyl-1H-pyrazol-3-yl)amino)-1-oxopropan-2-yl)phenyl)-4- |
| | (dimethylamino)but-2-enamide; |
| 121 | 2-(1-((5-cyclopropyl-1H-pyrazol-3-yl)amino)-1-oxopropan-2-yl)phenyl (E)-4-(4- |
| | (dimethylamino)but-2-enoyl)piperazine-1-carboxylate; |
| 122 | 4-(1-((5-cyclopropyl-1H-pyrazol-3-yl)amino)-1-oxopropan-2-yl)phenyl (E)-4-(4- |
| | (dimethylamino)but-2-enoyl)piperazine-1-carboxylate; |
| 123 | (E)-N-(5-(3-(1-((5-cyclopropyl-1H-pyrazol-3-yl)amino)-1-oxopropan-2-yl)phenyl)- |
| | 6-fluoropyridin-2-yl)-4-(dimethylamino)but-2-enamide; |
| 124 | (E)-N-(3-(3-(1-((5-cyclopropyl-1H-pyrazol-3-yl)amino)-1-oxopropan-2- |
| | yl)phenyl)pyridin-4-yl)-4-morpholinobut-2-enamide; |
| 125 | (E)-N-(3′-(1-((5-cyclopropyl-1H-pyrazol-3-yl)amino)-1-oxopropan-2-yl)-[1,1′- |
| | biphenyl]-2-yl)-4-morpholinobut-2-enamide; |
| 126 | (E)-N-(3-(1-((5-cyclopropyl-1H-pyrazol-3-yl)amino)-1-oxopropan-2-yl)phenyl)-4- |
| | (4-(dimethylamino)but-2-enoyl)piperazine-1-carboxamide; |
| 127 | (E)-N-(6-(3-((S)-1-((5-cyclopropyl-1H-pyrazol-3-yl)amino)-1-oxopropan-2- |
| | yl)phenyl)pyridin-2-yl)-4-((S)-3-fluoropyrrolidin-1-yl)but-2-enamide; |
| 128 | (E)-N-(6-(3-((S)-1-((5-cyclopropyl-1H-pyrazol-3-yl)amino)-1-oxopropan-2- |
| | yl)phenyl)pyrazin-2-yl)-4-((S)-3-fluoropyrrolidin-1-yl)but-2-enamide; |
| 129 | (E)-N-(5-(3-((S)-1-((5-cyclopropyl-1H-pyrazol-3-yl)amino)-1-oxopropan-2- |
| | yl)phenyl)pyridin-2-yl)-4-((S)-3-(trifluoromethyl)pyrrolidin-1-yl)but-2-enamide; |
| 130 | (E)-4-((2S,4S)-2-cyano-4-fluoropyrrolidin-1-yl)-N-(5-(3-((S)-1-((5-cyclopropyl-1H- |
| | pyrazol-3-yl)amino)-1-oxopropan-2-yl)phenyl)pyridin-2-yl)but-2-enamide; |
| 131 | (E)-4-((S)-2-cyanopyrrolidin-1-yl)-N-(5-(3-((S)-1-((5-cyclopropyl-1H-pyrazol-3- |
| | yl)amino)-1-oxopropan-2-yl)phenyl)pyridin-2-yl)but-2-enamide; |
| 132 | (S,E)-N-(3′-(1-((5-cyclopropyl-1H-pyrazol-3-yl)amino)-1-oxopropan-2-yl)-3-fluoro- |
| | [1,1′-biphenyl]-4-yl)-4-(diethylamino)but-2-enamide; |
| 133 | (E)-N-(4-(3-((5-cyclopropyl-1H-pyrazol-3-yl)amino)-4-methylisoquinolin-6- |
| | yl)phenyl)-4-(dimethylamino)but-2-enamide; |
| 134 | (E)-N-(4-(4-(1-((5-cyclopropyl-1H-pyrazol-3-yl)amino)-1-oxopropan-2-yl)pyridin- |
| | 2-yl)phenyl)-4-(dimethylamino)but-2-enamide; |
| 135 | (E)-N-(4-(3-(1-((5-cyclopropyl-1H-pyrazol-3-yl)amino)-1-oxopropan-2- |
| | yl)phenyl)cyclohexyl)-4-(dimethylamino)but-2-enamide; |
| 136 | (E)-N-(3-(3-(1-((5-cyclopropyl-1H-pyrazol-3-yl)amino)-1-oxopropan-2- |
| | yl)phenyl)cyclohexyl)-4-(dimethylamino)but-2-enamide; |
| 137 | (E)-4-(dimethylamino)-N-(3′-(1-oxo-1-(pyrazolo[1,5-a]pyridin-2-ylamino)propan-2- |
| | yl)-[1,1′-biphenyl]-4-yl)but-2-enamide; |
| 138 | (E)-N-(3′-(3-((5-cyclopropyl-1H-pyrazol-3-yl)amino)-1,1,1-trifluoro-3-oxopropan- |
| | 2-yl)-[1,1′-biphenyl]-4-yl)-4-(dimethylamino)but-2-enamide; |
| 139 | (E)-N-(3′-(1-((5-cyclopentyl-1H-pyrazol-3-yl)amino)-1-oxopropan-2-yl)-[1,1′- |
| | biphenyl]-4-yl)-4-(dimethylamino)but-2-enamide; |
| 140 | (E)-N-(3′-(1-((5-cyclohexyl-1H-pyrazol-3-yl)amino)-1-oxopropan-2-yl)-[1,1′- |
| | biphenyl]-4-yl)-4-(dimethylamino)but-2-enamide; |
| 141 | (E)-N-(3′-(1-((5-cyclopropyl-4-methyl-1H-pyrazol-3-yl)amino)-1-oxopropan-2-yl)- |
| | [1,1′-biphenyl]-4-yl)-4-(dimethylamino)but-2-enamide; |
| 142 | (E)-N-(3′-(1-((5-cyclopropyl-4-(pyridin-2-yl)-1H-pyrazol-3-yl)amino)-1-oxopropan- |
| | 2-yl)-[1,1′-biphenyl]-4-yl)-4-(dimethylamino)but-2-enamide; and |
| 143 | (E)-N-(3′-(3-(5-cyclopropyl-1H-pyrazol-3-yl)-1-methylureido)-[1,1′-biphenyl]-4- |
| | yl)-4-(dimethylamino)but-2-enamide; |

or a pharmaceutically acceptable salt thereof or a stereoisomer thereof.

19.-20. (canceled)

21. The method of claim 1, wherein the compound of formula (I) is (E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide or a pharmaceutically acceptable salt thereof or a stereoisomer thereof.

22. The method of claim 1, wherein the compound of formula (I) is (S)-(E)-N-(5-(3-(1-((5-cyclopropyl-1H-pyrazol-3-yl)amino)-3-methyl-1-oxobutan-2-yl)phenyl)pyridin-2-yl)-4-morpholinobut-2-enamide or a pharmaceutically acceptable salt thereof.

23. (canceled)

24. The method of claim 22, wherein the cancer is prostate cancer.

25. The method of claim 24, wherein the prostate cancer is metastatic castration-resistant prostate cancer.

26. The method of claim 22, wherein the cancer is selected from the group consisting of breast cancer, head and neck cancer, squamous cell carcinoma, stomach cancer, ovarian cancer, soft-tissue sarcoma, adenocarcinoma, lung cancer and non-small-cell lung cancer.

27. The method of claim 22, wherein the anti-microtubule agent is docetaxel.

28.-30. (canceled)
